(19) United States
(12) Patent Application Publication
(10) Pub. No.: US 2026/0028737 A1
(43) Pub. Date: Jan. 29, 2026

(54) METHODS AND SYSTEMS FOR POWER-TO-FUEL APPLICATIONS USING TAIL GAS FROM FUEL SYNTHESIS AND/OR METHANE IN HIGH TEMPERATURE ELECTROLYZER

(71) Applicant: Chevron U.S.A. Inc., San Ramon, CA (US)

(72) Inventors: Zihan Huang, Emeryville, CA (US); Lin Li, San Ramon, CA (US); Oliver Yair Gutiérrez-Tinoco, Martinez, CA (US); Tapan Das, Albany, CA (US); Evan Michael Visser, Spring, TX (US)

(21) Appl. No.: 19/277,803

(22) Filed: Jul. 23, 2025

Related U.S. Application Data
(60) Provisional application No. 63/674,983, filed on Jul. 24, 2024.

(51) Int. Cl. C25B 15/08; C07C 29/152; C07C 41/01; C25B 1/042; C25B 9/70

(52) U.S. Cl. CPC C25B 15/081; C07C 29/152; C07C 41/01; C25B 1/042; C25B 9/70; C25B 15/087

(57) ABSTRACT

A continuous method includes passing a first steam feed stream to a cathode of an electrolyzer including the cathode, an anode and an electrolyte inserted between the cathode and the anode, thereby producing a cathode effluent including hydrogen, passing a second steam feed stream and one or more of a recycled tail gas stream from a reactor unit and a methane-rich feed stream to the anode of the electrolyzer, wherein the one or more of the recycled tail gas stream and the methane-rich feed stream are utilized as fuel for producing the cathode effluent including hydrogen, and passing the cathode effluent including g hydrogen and a carbon dioxide feed stream to the reactor unit, thereby producing a chemical product or a fuel-based product.

### PRIORITY CLAIM

The present application claims priority to U.S. Provisional Patent Application No. 63/674,983, entitled “Methods and Systems for Power-to-Fuel Applications Using Tail Gas from Fuel Synthesis and/or Methane in High Temperature Electrolyzer,” filed Jul. 24, 2024, the content of which is incorporated by reference herein in its entirety.

### BACKGROUND

Many energy production processes release carbon dioxide. In order to reduce the climate-damaging effects of carbon dioxide in the atmosphere, the carbon dioxide can be converted into other substances. However, this is made more difficult by the fact that carbon dioxide is stable and not very reactive. In addition, the carbon in the carbon dioxide molecule is in its highest oxidation state. Thus, carbon dioxide can no longer be used to generate energy by means of incineration.

Carbon dioxide can also be reduced to liquid hydrocarbons. For example, carbon dioxide can be directly hydrogenated to methane in the presence of a catalyst.

Producing a chemical using electricity is also known as “Power-to-X” because electricity (“power”) can be used to obtain a chemical (“X”). By using climate-damaging carbon dioxide as a starting material, this concept can contribute to reducing global warming.

### SUMMARY

In accordance with an illustrative embodiment, a continuous method comprises:
passing a first steam feed stream to a cathode of an electrolyzer comprising the cathode, an anode and an electrolyte inserted between the cathode and the anode, thereby producing a cathode effluent comprising hydrogen,
passing a second steam feed stream and one or more of a recycled tail gas stream from a reactor unit and a methane-rich feed stream to the anode of the electrolyzer, wherein the one or more of the recycled tail gas stream and the methane-rich feed stream are utilized as fuel for producing the cathode effluent comprising hydrogen, and
passing the cathode effluent comprising hydrogen and a carbon dioxide feed stream to the reactor unit, thereby producing a chemical product or a fuel-based product.

In accordance with another illustrative embodiment, a continuous method comprises:
passing a steam and carbon dioxide feed stream to a cathode of an electrolyzer comprising the cathode, an anode and an electrolyte inserted between the cathode and the anode, thereby producing a cathode effluent comprising syngas,
passing a steam feed stream and one or more of a recycled tail gas stream from a reactor unit and a methane-rich feed stream to the anode of the electrolyzer, wherein the one or more of the recycled tail gas stream and the methane-rich feed stream are utilized as fuel for producing the cathode effluent comprising syngas, and
passing the cathode effluent comprising syngas to the reactor unit, thereby producing a chemical product or a fuel-based product.

In accordance with yet another illustrative embodiment, a system comprises:
an electrolyzer comprising a cathode, an anode and an electrolyte inserted between the cathode and the anode, wherein the electrolyzer is configured to generate, in the cathode, one of hydrogen from a steam feed stream or syngas from a steam can carbon dioxide feed stream; wherein the electrolyzer is further configured to receive one or more of a recycled tail gas stream and a methane-rich feed stream in the anode utilized as fuel for producing the hydrogen or the syngas in the cathode; and
a reactor unit configured to generate a chemical product or a fuel-based product from the hydrogen or the syngas and a carbon dioxide feed stream.

### BRIEF DESCRIPTION OF THE DRAWINGS

In combination with the accompanying drawings and with reference to the following detailed description, the features, advantages, and other aspects of the implementations of the present disclosure will become more apparent, and several implementations of the present disclosure are illustrated herein by way of example but not limitation. In the accompanying drawings:

FIG. 1A illustrates a method and system diagram scheme utilizing a first high-temperature solid-oxide electrolyzer for steam electrolysis of a steam feed stream in the cathode to produce hydrogen and a second high-temperature solid-oxide electrolyzer for steam electrolysis of a steam feed stream in the cathode to produce hydrogen, and a recycled tail gas stream in the anode to produce syngas via a reforming reaction and a partial oxidation reaction in the anode as an energy source for electrolysis, for sending to a downstream synthesis reactor for production of a chemical product or fuel, according to an illustrative embodiment.

FIG. 1B illustrates a method and system diagram scheme utilizing a first high-temperature solid-oxide electrolyzer for steam electrolysis of a steam feed stream in the cathode to produce hydrogen and a second high-temperature solid-oxide electrolyzer for steam electrolysis of a steam feed stream in the cathode to produce hydrogen, and a recycled tail gas stream and a methane-rich feed stream in the anode to produce syngas via a reforming reaction and a partial oxidation reaction in the anode as an energy source for electrolysis, for sending to a downstream synthesis reactor for production of a chemical product or fuel, according to an illustrative embodiment.

FIG. 1C illustrates a method and system diagram scheme utilizing a first high-temperature solid-oxide electrolyzer for steam electrolysis of a steam feed stream in the cathode to produce hydrogen in the cathode and a second high-temperature solid-oxide electrolyzer for steam electrolysis of a steam feed stream in the cathode to produce hydrogen, and a methane-rich feed stream in the anode to produce syngas via a reforming reaction and a partial oxidation reaction in the anode as an energy source for electrolysis, for sending to a downstream synthesis reactor for production of a chemical product or fuel, according to an illustrative embodiment.

FIG. 2 is a schematic view showing a high-temperature steam electrolysis solid-oxide electrolyzer (SOEC), according to an illustrative embodiment.

FIG. 3A illustrates a method and system diagram scheme utilizing a first high-temperature solid-oxide electrolyzer for co-electrolysis of a steam and carbon dioxide feed stream in the cathode to produce syngas and a second high-temperature solid-oxide electrolyzer for co-electrolysis of a steam and carbon dioxide feed stream in the cathode to produce syngas, and a recycled tail gas stream in the anode to produce syngas via a reforming reaction and a partial oxidation reaction in the anode as an energy source for electrolysis, for sending to a downstream synthesis reactor for production of a chemical product or fuel, according to an alternative illustrative embodiment.

FIG. 3B illustrates a method and system diagram scheme utilizing a first high-temperature solid-oxide electrolyzer for co-electrolysis of a steam and carbon dioxide feed stream in the cathode to produce syngas and a second high-temperature solid-oxide electrolyzer for co-electrolysis of a steam and carbon dioxide feed stream in the cathode to produce syngas, and a recycled tail gas stream and a methane-rich feed stream in the anode to produce syngas via a reforming reaction and a partial oxidation reaction in the anode as an energy source for electrolysis, for sending to a downstream synthesis reactor for production of a chemical product or fuel, according to an alternative illustrative embodiment.

FIG. 3C illustrates a method and system diagram scheme utilizing a first high-temperature solid-oxide electrolyzer for co-electrolysis of a steam and carbon dioxide feed stream in the cathode to produce syngas and a second high-temperature solid-oxide electrolyzer for co-electrolysis of a steam and carbon dioxide feed stream in the cathode to produce syngas, and a methane-rich feed stream in the anode to produce syngas via a reforming reaction and a partial oxidation reaction in the anode as an energy source for electrolysis, for sending to a downstream synthesis reactor for production of a chemical product or fuel, according to an alternative illustrative embodiment.

FIG. 4A illustrates a method and system diagram scheme utilizing a first high-temperature solid-oxide electrolyzer for co-electrolysis of a steam and carbon dioxide feed stream in the cathode to produce syngas and a second high-temperature solid-oxide electrolyzer for steam electrolysis of a steam feed stream in the cathode to produce hydrogen, and a recycled tail gas stream in the anode to produce syngas via a reforming reaction and a partial oxidation reaction in the anode as an energy source for electrolysis, for sending to a downstream synthesis reactor for production of a chemical product or fuel, according to an illustrative embodiment.

FIG. 4B illustrates a method and system diagram scheme utilizing a first high-temperature solid-oxide electrolyzer for co-electrolysis of a steam and carbon dioxide feed stream in the cathode to produce syngas and a second high-temperature solid-oxide electrolyzer for steam electrolysis of a steam feed stream in the cathode to produce hydrogen, and a recycled tail gas stream and a methane-rich feed stream in the anode to produce syngas via a reforming reaction and a partial oxidation reaction in the anode as an energy source for electrolysis, for sending to a downstream synthesis reactor for production of a chemical product or fuel, according to an illustrative embodiment.

FIG. 4C illustrates a method and system diagram scheme utilizing a first high-temperature solid-oxide electrolyzer for co-electrolysis of a steam and carbon dioxide feed stream in the cathode to produce syngas and a second high-temperature solid-oxide electrolyzer for steam electrolysis of a steam feed stream in the cathode to produce hydrogen, and a methane-rich feed stream in the anode to produce syngas via a reforming reaction and a partial oxidation reaction in the anode as an energy source for electrolysis, for sending to a downstream synthesis reactor for production of a chemical product or fuel, according to an illustrative embodiment.

FIG. 5A illustrates a method and system diagram scheme utilizing a high-temperature solid-oxide electrolyzer for steam electrolysis of a steam feed stream in the cathode to produce hydrogen for combining with carbon dioxide for sending to a downstream synthesis reactor for production of chemical products or fuels using a recycled tail gas stream and its partial oxidation in the anode as an energy source for electrolysis, according to an illustrative embodiment.

FIG. 5B illustrates a method and system diagram scheme utilizing a high-temperature solid-oxide electrolyzer for steam electrolysis of a steam feed stream in the cathode to produce hydrogen for combining with carbon dioxide for sending to a downstream synthesis reactor for production of chemical products or fuels using a recycled tail gas stream and a methane-rich feed stream and its partial oxidation in the anode as an energy source for electrolysis, according to an illustrative embodiment.

FIG. 5C illustrates a method and system diagram scheme utilizing a high-temperature solid-oxide electrolyzer for steam electrolysis of a steam feed stream in the cathode to produce hydrogen for combining with carbon dioxide for sending to a downstream synthesis reactor for production of chemical products or fuels using a methane-rich feed stream and its partial oxidation in the anode as an energy sources energy for electrolysis, according to an illustrative embodiment.

FIG. 6A illustrates a method and system diagram scheme utilizing a high-temperature solid-oxide electrolyzer for co-electrolysis of a steam and carbon dioxide feed stream in the cathode to produce syngas using a recycled tail gas stream and its partial oxidation in the anode as an energy source for electrolysis, according to an illustrative embodiment.

FIG. 6B illustrates a method and system diagram scheme utilizing a high-temperature solid-oxide electrolyzer for co-electrolysis of a steam and carbon dioxide feed stream in the cathode to produce syngas using a recycled tail gas stream and a methane-rich feed stream and its partial oxidation in the anode as an energy source for electrolysis, according to an illustrative embodiment.

FIG. 6C illustrates a method and system diagram scheme utilizing a high-temperature solid-oxide electrolyzer for co-electrolysis of a steam and carbon dioxide feed stream in the cathode to produce syngas using a methane-rich feed stream and its partial oxidation in the anode as an energy source for electrolysis, according to an illustrative embodiment.

### DETAILED DESCRIPTION

Various illustrative embodiments described herein are directed to methods and systems for the electrochemical conversion of one or more of a recycled tail gas stream from a reactor unit and a methane-rich feed stream into syngas for the production of chemical products and/or fuel-based products.

“Power-to-gas,” “power-to-liquid,” and “power-to-fuel” (referred to as “Power-to-X” processes) represent promising approaches for bringing about a future conversion from fossil energy sources to an energy infrastructure which is based mainly on renewable energy sources (RES), for example wind power, solar power, geothermal energy, water power or hydroelectric power. Electricity-based fuels or synthetic fuels are becoming ever more important, particularly in the transport sector or in industry. Such fuels, for example methane, methanol or derivatives or downstream products such as kerosene, gasoline, diesel, or other hydrocarbon-based products are produced, in particular, by synthesis from hydrogen and carbon dioxide.

Power-to-X processes utilize electrical energy to convert carbon dioxide into carbon-neutral fuels or chemicals. For example, it is possible to convert carbon dioxide and water directly into multi-carbon species as desired fuels or chemicals. However, such a direct carbon conversion route for fuel synthesis is still at the early stage of development and faces significant technical challenges. On the other hand, the indirect route involves carbon dioxide and water being first converted into hydrogen and carbon monoxide by co-electrolysis, which can be further transformed into desired products in a downstream synthesis reaction process, e.g., an electrolyzer by way of co-electrolysis can convert water and carbon dioxide to syngas, and the produced syngas can be further reacted to make desired chemical products or fuel-based products.

For example, the Fisher-Tropsch (FT) process can convert syngas to liquid fuels or base oils. In addition to the desired liquid hydrocarbon products, the FT process also produces a significant undesirable light gas fraction (e.g., C₁ to C₄ products). The gaseous light products, leaving the synthesis reactor as a tail gas, are less valuable than liquid products, more difficult to transport, and it is not practical to sell them as products for some applications. For conventional GTL (natural gas to liquid) plants, the tail gas is typically used as fuel gas. For CO₂ conversion through Power-to-X, it is undesirable to use tail gas as fuel. Burning of tail gas will produce CO₂, which will reduce the net CO₂ utilization rate (i.e., the percentage of total CO₂ feed converted to target liquid products). Therefore, there is a need to better utilize the tail gas while improving the overall energy efficiency and CO₂ utilization for a practicable CO₂ to chemical products or fuels process.

In this context of FT fuel synthesis, methane (CH₄) and other light hydrocarbons present in the tail gas of the fuel synthesis can be recycled back to the anode of the SOEC. The use of methane in this manner has the potential to enhance the energy efficiency of the system. This is achieved by reducing the electrical power demand of the SOEC, making the process more cost-effective. Firstly, CH₄ (and other light hydrocarbons) can act as a fuel source, such as by providing heat to the system through the exothermic partial oxidation reaction as follows.

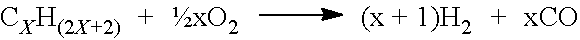

This heat can assist in maintaining the high operating temperature of the SOEC, thereby reducing the need for external heating.

Secondly, these hydrocarbons depolarize the anode by reacting with oxygen ions in the anode to generate electrons, effectively lowering the cell voltage and reducing the electrical power consumption of the SOEC compared to no methane (or light hydrocarbon) recycle in the anode. In addition, Steam Methane Reforming (SMR) can occur with a steam feed to the anode to further enhance the energy efficiency of the system. In SMR, methane reacts with steam to produce syngas (hydrogen and carbon monoxide) as follows.:

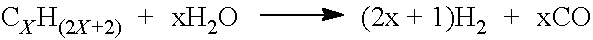

The heat required for this endothermic reaction can be supplied by the heat generated from the partial oxidation of methane in the SOEC. The hydrogen and carbon monoxide produced by SMR can then be used in the synthesis of fuels and chemicals, further reducing the overall energy consumption of the system.

Methanol synthesis from CO₂ is an attractive option for CO₂ utilization because methanol is a liquid fuel that is relatively easy to transport with numerous applications. Although direct CO₂ and hydrogen to methanol conversion technology is available, the slower reaction kinetics result in a significantly lower per-pass carbon conversion and therefore much larger reactors are required. By incorporating SOEC co-electrolysis, the resulting syngas product allows for traditional methanol synthesis, and also provides a destination for any light hydrocarbon by-products. Additionally, renewable natural gas (biomethane) can be used as a feedstock to the electrolysis process, particularly for methanol and dimethyl ether synthesis. By introducing methane, it is possible to reduce the electricity demand as previously mentioned while maintaining a consistent hydrogen to carbon ratio of the syngas product, as preferred for downstream synthesis. This is especially attractive at times when renewable electricity is less available, thereby significantly increasing the overall flexibility of the system and the diversification of feedstock.

Accordingly, the non-limiting illustrative embodiments disclosed herein overcome the foregoing drawbacks by utilizing the recycling of a tail gas stream from downstream fuel synthesis and/or a methane-rich feed stream to an SOEC electrolysis to enhance the overall energy efficiency and carbon dioxide utilization rate for converting carbon dioxide to chemical products and/or fuel-based products. The non-limiting illustrative embodiments described herein are able to maximize the utilization of the available energy from the recycled tail gas stream and/or methane-rich feed stream being sent to an electrolyzer, thereby reducing the electrolyzer electricity consumption while improving energy efficiency and reducing electricity cost.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

While systems and methods are described in terms of “comprising” various components or steps, the systems and methods can also “consist essentially of” or “consist of” the various components or steps, unless stated otherwise.

The terms “a,” “an,” and “the” are intended to include plural alternatives, e.g., at least one. The terms “including,” “with,” and “having,” as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means ±20% of the stated value, ±15% of the stated value, ±10% of the stated value, ±5% of the stated value, ±3% of the stated value, or ±1% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

The term “continuous” as used herein shall be understood to mean a system that operates without interruption or cessation for a period of time, such as where at least reactant(s) are continually fed into a reaction zone of a reactor unit and products are continually or regularly withdrawn without stopping the reaction in the reaction zone.

The term “reactor unit product-forming feed stream” as used herein shall be understood to mean a feed stream which contains hydrogen and at least one of carbon monoxide and carbon dioxide such that when passed into the reactor unit one or more of a chemical product and/or fuel can be produced as discussed below. As one skilled in the art will understand, the hydrogen can be formed from one or more of steam electrolysis in one or more electrolyzers described herein, co-electrolysis of a steam feed stream and a carbon dioxide feed stream in one or more electrolyzers, and a reforming reaction of a steam feed stream and one or more of a recycled tail gas stream from a reactor unit and a methane-rich feed stream and its partial oxidation in one or more electrolyzers. The carbon monoxide can be formed from one or more of co-electrolysis of a steam feed stream and a carbon dioxide feed stream in one or more electrolyzers, and a reforming reaction of a steam feed stream and one or more of a recycled tail gas stream from a reactor unit and a methane-rich feed stream and its partial oxidation in one or more electrolyzers.

The term “electrode” is meant as, in the sense of the present disclosure, an electronic conductor capable of capturing or releasing electrons. An oxidation reaction occurs at the anode, whereas a reduction reaction occurs at the cathode.

The term “electrolysis” is a technique that uses direct electric current to drive an otherwise non-spontaneous chemical reaction. For example, the electrolysis of water is the process of using electricity to electrochemically decompose water into oxygen and hydrogen. The term “electrolyzer,” also called an “electrolysis device,” refers to a unit where this chemical reaction may take place.

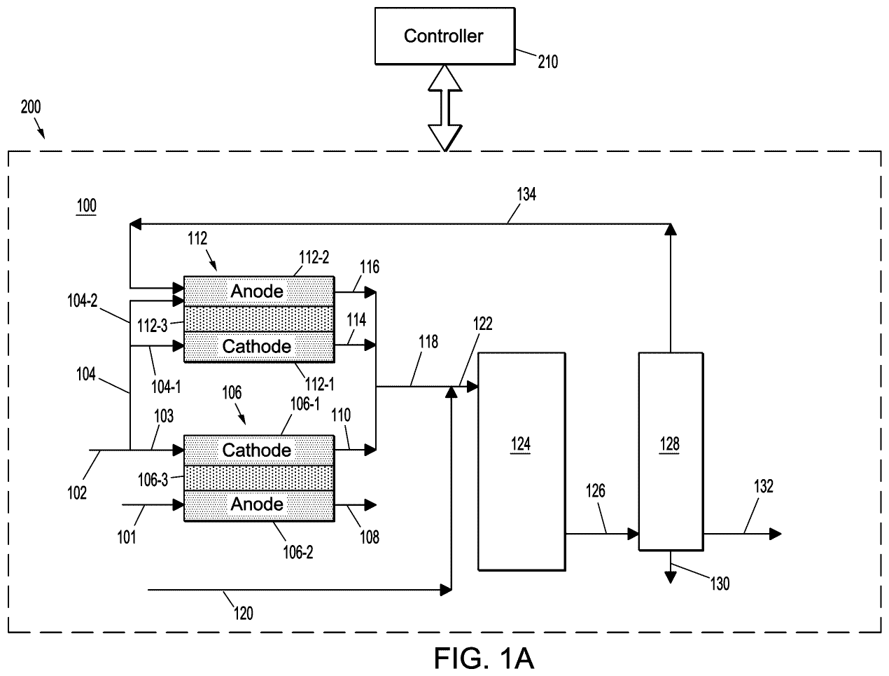

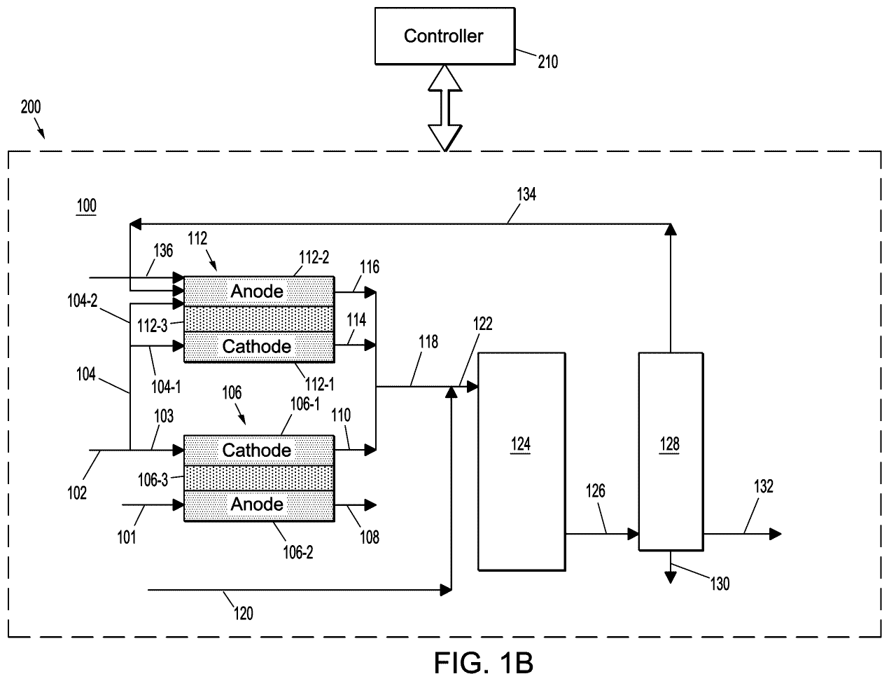

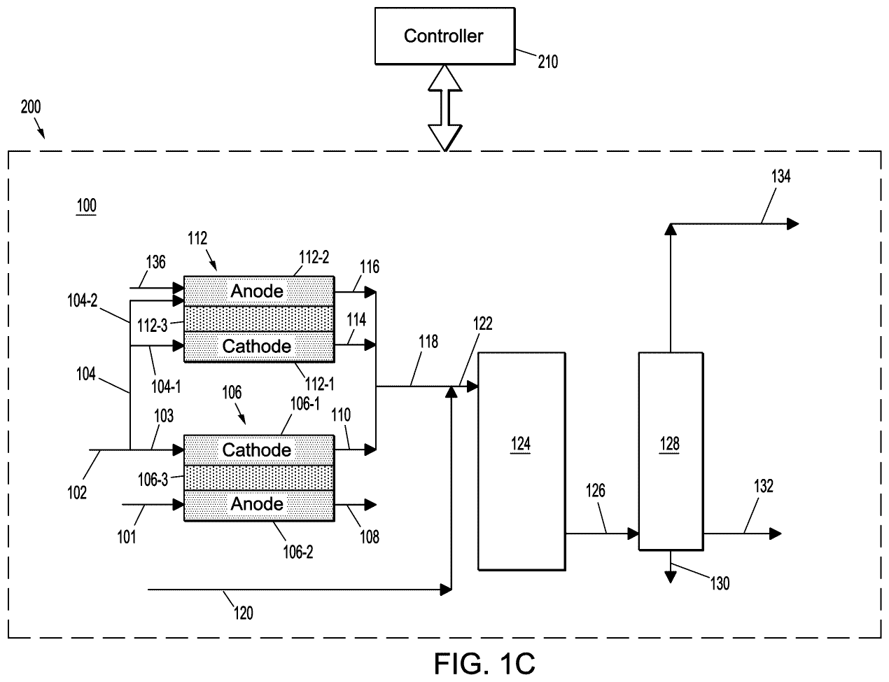

The illustrative embodiments of the present disclosure will be specifically described below with reference to the accompanying drawings. For the purpose of clarity, some steps leading up to the production of syngas through electrochemical conversion of one or more of a recycled tail gas stream from a reactor unit and a methane-rich feed stream in an anode of an electrolyzer and/or steam and carbon dioxide in a cathode of an electrolyzer to chemical products and/or fuel-based products as illustrated in FIGS. 1A-6C are omitted. In other words, one or more well-known processing steps which are not illustrated but are well-known to those of ordinary skill in the art have not been included in the figures. This is not intended to be interpreted as a limitation of any particular embodiment, or illustration, or scope of the claims.

FIGS. 1A-1C illustrate a method and system diagram scheme utilizing a first high-temperature solid-oxide electrolyzer for electrolysis of a steam feed stream in the cathode to produce hydrogen and a second high-temperature solid-oxide electrolyzer for steam electrolysis of a steam feed stream in the cathode to produce hydrogen, and a steam feed and one or more of a recycled tail gas stream and a methane-rich feed stream in the anode to produce syngas via a reforming reaction and a partial oxidation reaction in the anode as an energy source for electrolysis.

Referring now to FIGS. 1A-IC, an anode purge stream 101 and a first steam feed stream 102 are sent to a system 100. Anode purge stream 101 includes one or more of air, carbon dioxide or an inert gas such as N₂. First steam feed stream 102 can be obtained from any means known in the art and is split into a second steam feed stream 103 and a third steam feed stream 104.

In some embodiments, anode purge stream 101 such as air may be pressurized to produce a pressurized anode purge stream using fans, blowers, compressors or a combination thereof (not shown). The air compressor may be centrifugal, mixed-flow, axial-flow, reciprocating, rotary screw, rotary vane, scroll, diaphragm compressor, or a combination thereof. In some embodiments, the pressurized anode purge stream may have a pressure between about 1 bar to about 20 bar. In some embodiments, the pressurized anode purge stream may have a pressure of about 1 bar, about 1.2 bar, about 5 bar, about 10 bar, about 15 bar, or about 20 bar. In some embodiments, the pressurized anode purge stream may have a pressure of from about 1 bar to about 3 bar, e.g., about 1 bar, about 1.2 bar, about 1.4 bar, about 1.6 bar, about 1.8 bar, about 2.0 bar, about 2.2 bar, about 2.4 bar, about 2.6 bar, about 2.8 bar or about 3 bar.

System 100 includes a first electrolyzer 106 comprising a cathode 106-1, an anode 106-2 and an electrolyte 106-3 inserted between cathode 106-1 and anode 106-2. First electrolyzer 106 receives anode purge stream 101 into anode 106-2 and second steam feed stream 103 into cathode 106-1 where second steam feed stream 103 is converted to a cathode effluent 110 composed mainly of H₂ having a temperature of about 750° C. to about 850° C. from cathode 106-1 and an anode effluent 108 which is an oxygen enriched stream from anode 106-2. Anode purge stream 101 serves as a purge gas to carry oxygen generated at anode 106-2. Anode effluent 108 can be discarded or recycled back to first electrolyzer 106. In an illustrative embodiment, first electrolyzer 106 can be any suitable high temperature electrolyzer comprising cathode 106-1, anode 106-2 and electrolyte 106-3 inserted between cathode 106-1 and anode 106-2. In a non-limiting illustrative embodiment, first electrolyzer 106 is a high temperature solid oxide electrolyzer (also referred to as SOEC) for steam electrolysis comprising:
a first porous conductive electrode, or “cathode,” to be supplied with steam for the production of dihydrogen,
a second porous conductive electrode, or “anode,” via which the dioxygen (O₂) produced by the electrolysis of the water injected onto the cathode escapes, and
a solid oxide membrane (dense electrolyte) sandwiched between the cathode and the anode, the membrane being anionically conductive at high temperatures, usually temperatures above about 700° C. and up to about 950° C.

First electrolyzer 106 may receive input energy (i.e., electricity) from an intermittent source such as solar power (including photovoltaic and reflective), wind power, tidal power, wave power, batteries, and other intermittent energy sources known in the art and combinations thereof. Alternatively, or in addition, first electrolyzer 106 may receive input energy from a non-intermittent source, such as an electricity grid (e.g., a regional electricity grid, a municipal electricity grid, or a microgrid), natural gas, coal, nuclear, and other non-intermittent sources known in the art and combinations thereof. First electrolyzer 106 may therefore be electricity connectable to an intermittent energy input, a non-intermittent source, or a combination thereof. In particular embodiments, first electrolyzer 106 may receive input energy from the photovoltaic panel.

In some embodiments, first electrolyzer 106 may be operational by receiving electricity from a photovoltaic panel. At night, first electrolyzer 106 may be operated in “hot standby” mode to conserve electricity, or first electrolyzer 106 may be electricity connected to another power source to continue operating at night. In particular, first electrolyzer 106 may be connected to a power grid such as a regional power grid, a municipal power grid, or a micro grid, and first electrolyzer 106 may run when the price of electricity is low.

System 100 may further comprise an energy storage mechanism or a plurality of energy storage mechanisms. The energy storage mechanism may comprise any mechanism or apparatus operable to store energy such as electricity, thermal energy, etc. For example, the energy storage mechanism may include batteries (e.g., lead-acid batteries, lithium-ion batteries, lithium-ion batteries, etc.), water, flywheels, compressed air, pumped hydroelectric, or other energy storage mechanisms known in the art and combinations thereof.

As an overview, for high-temperature steam electrolysis (HTSE), steam (H₂O) is injected into at least the cathode compartment of the electrolyzer. Under the effect of the electrical current applied to the cell, the dissociation of water molecules in the form of steam occurs at the interface between the hydrogen electrode (cathode) and the electrolyte, where this dissociation produces dihydrogen gas (H₂) and oxygen ions (O²⁻). Dihydrogen (H₂) is collected and discharged at the outlet of the hydrogen compartment. The oxygen ions (O²⁻) migrate through the electrolyte and form dioxygen (O₂) at the interface between the electrolyte and the oxygen electrode (anode). A draining gas, such as air, can circulate at the anode and thus collect the oxygen generated in gas form at the anode.

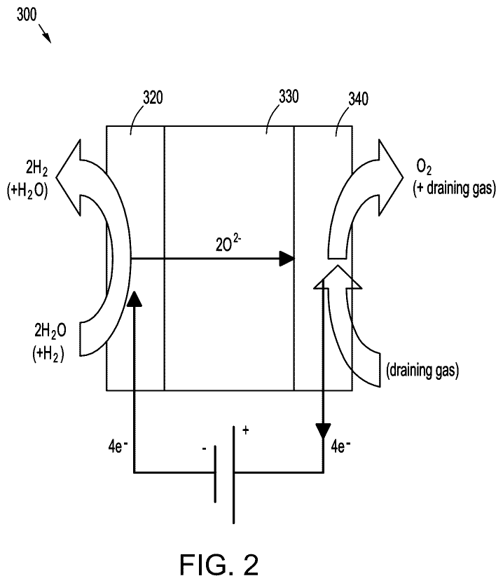

In an illustrative embodiment, FIG. 2 shows a schematic view of the principle of operation of a high-temperature solid-oxide electrolyzer (SOEC). Such an electrolyzer is an electrochemical device for producing hydrogen (and oxygen) under the effect of an electrical current. In these electrolyzers, the high-temperature electrolysis of water is performed using steam. Thus, the function of such an electrolyzer is to transform the steam into hydrogen and oxygen according to the following chemical reaction:

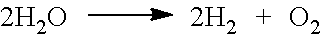

This reaction occurs electrochemically in the cells of the electrolyzer. As schematically shown in FIG. 2, each basic electrolysis cell 300 is formed by a cathode 320 and an anode 340, placed on either side of an electrolyte 330. The two electrodes (i.e., cathode 320 and anode 340) are electronic and/or ionic conductors, made of porous material, and electrolyte 330 is impervious to gas, an electronic insulator and an ion conductor. Electrolyte 330 may in particular be an anion conductor, and more specifically an anion conductor of O²⁻ions, and the electrolyzer is then referred to as an anion electrolyzer, by contrast with proton electrolytes (H⁺).

The electrochemical reactions occur at the interface between each of the electronic conductors and the ion conductor.

At cathode 320, the half-reaction is as follows:

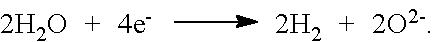

At the anode 340, the half-reaction is as follows:

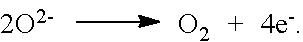

Electrolyte 330, inserted between the two electrodes, i.e., cathode 320 and anode 340, is the site of migration of the O²⁻ions under the effect of the electrical field created by the difference in potential imposed between anode 340 and cathode 320.

As indicated between parentheses in FIG. 2, the steam at the cathode inlet can be accompanied by hydrogen H₂ and the hydrogen produced and recovered at the outlet can be accompanied by steam. Similarly, a draining gas, such as air, may also be injected at the inlet to discharge the oxygen produced. The injection of a draining gas has the additional function of acting as a temperature controller.

A basic electrolyzer, or electrolysis reactor, therefore consists of a basic cell as described above, with cathode 320, electrolyte 330, and anode 340, and two monopolar connectors, which provide electrical, hydraulic and thermal distribution functions.

To increase the flow rates of hydrogen and oxygen produced, a stack of a plurality of basic electrolysis cells one on top of another can be used, separating them with interconnection devices, usually called interconnectors or bipolar interconnection plates. The assembly is positioned between two end interconnection plates that support the electrical and gas supplies of the electrolyzer (electrolysis reactor).

A high-temperature solid-oxide electrolyzer (SOEC) thus comprises at least one, and generally a plurality of electrolysis cells stacked one on top of another, each basic cell being formed by an electrolyte, a cathode and an anode, the electrolyte being inserted between the cathode and the anode.

Thus, the function of a so-called cathode compartment is to distribute the electrical current and steam as well as to recover hydrogen at the cathode in contact.

The function of a so-called anode compartment is to distribute the electrical current and to recover oxygen at the anode in contact, optionally by means of a draining gas.

Turning back to FIGS. 1A-1C, in some embodiments, second steam feed stream 103 is converted to cathode effluent 110 composed mainly of hydrogen (H₂), and anode effluent 108 composed mainly of oxygen (oxygen enriched stream) each having a temperature of about 750°° C. to about 850° C. in first electrolyzer 106 (i.e., a solid oxide electrolytic cell (SOEC)). For example, second steam feed stream 103 is converted to cathode effluent 110 composed mainly of hydrogen (H₂) at cathode 106-1 in first electrolyzer 106 and anode purge stream 101 may participate as a purge gas to purge anode 106-2 of first electrolyzer 106, where cathode 106-1 and anode 106-2 may be separated by electrolyte 106-3. In some embodiments, cathode 106-1 may operate at a temperature between about 750°° C. to about 850° C., and the anode may operate at a temperature between about 750° C. to about 850° C.

In some embodiments, first electrolyzer 106 may operate at a temperature of about 700° C. to about 950° C. In some embodiments, first electrolyzer 106 may operate at a temperature of about 700° C., about 720°° C., about 730° C., about 740° C., about 750° C., about 760° C., about 770° C., about 780°° C., about 790°° C., about 800° C., about 810° C., about 820° C., about 830° C., about 840° C., about 860° C., about 880°° C., about 900°° C., about 910° C., about 920° C., about 930° C., about 940° C., or about 950° C., where any range from these limits are contemplated herein. In some embodiments, cathode 106-1 may operate at a temperature between about 750° C. to about 850° C., and anode 106-2 may operate at a temperature between about 750°° C. to about 850° C.

In some embodiments, first electrolyzer 106 may operate at a pressure between about 1 bar to about 20 bars. In some embodiments, first electrolyzer 106 may operate at a pressure of about 1.02 bar, about 3 bar, about 5 bar, about 7 bar, about 9 bar, about 10 bar, about 15 bar or about 20 bar, where any range from these limits are contemplated herein. In some embodiments, first electrolyzer 106 may operate at a pressure of about 1 bar to about 3 bar, e.g., about 1 bar, about 1.2 bar, about 1.4 bar, about 1.6 bar, about 1.8 bar, about 2.0 bar, about 2.2 bar, about 2.4 bar, about 2.6 bar, about 2.8 bar or about 3 bar, where any range from these limits are contemplated herein.

The material of the solid oxide electrolyzer electrodes (i.e., cathode 106-1 and anode 106-2) may be based on ceramic materials that exhibit stability through reduction-oxidation (redox) cycles, electrocatalytic activity, and mixed ionic and electronic conductivity in reducing atmospheres. The material of the solid oxide electrodes may be metal or metal oxide-based material (e.g., Ni-based electrodes). In some embodiments, the cathode and anode may be constructed of any suitable material including, for example, (La,Sr)(Fe,Co)O₃(LSCF), (Sm,Sr)CoO₃, and Sr-doped LaMnO₃ for the anode electrode (anode) and Ni-YSZ, Ni-ScSZ, La₂NiO₄, and Ni-ZrO₂ for the cathode electrode. Electrode support materials and functional layers include nickel cermets, and other electronic conductors such as (Sr_{0.8}La_{0.2})TiO₃(SLT).

Electrolyte 106-3 may be comprised of any suitable material such as, for example, yttria-stabilized zirconia (YSZ), (La_{0.6}Sr_{0.4}) (Ga_{0.8}Mg_{0.2})O₃(LSGM), Sc-stabilized zirconia (SSZ), and doped ceria. A SOEC cell architecture includes both electrode-and electrolyte-supported cell constructions and ceramic or metallic interconnects. It is to be understood that the above materials are merely exemplary and any known materials for use in the SOEC cell architecture are contemplated.

Referring back to third steam feed stream 104, third steam feed stream 104 is split into a fourth steam feed stream 104-1 and a fifth steam feed stream 104-2.

System 100 further includes a second electrolyzer 112 comprising a cathode 112-1, an anode 112-2 and an electrolyte 112-3 inserted between cathode 112-1 and anode 112-2. Second electrolyzer 112 receives fourth steam feed stream 104-1 into cathode 112-1 and fifth steam feed stream 104-2, and one or more of a recycled tail gas stream 134 from a separation unit 128 as discussed below and a methane-rich feed stream 136 into anode 112-2.

In some embodiments, as depicted in FIG. 1A, second electrolyzer 112 receives fifth steam feed stream 104-2 and recycled tail gas stream 134 from separation unit 128 containing methane and light hydrocarbons (e.g., CₓH₍₂ₓ₊₂₎ such as C₂ to C₄) into anode 112-2. Recycled tail gas stream 134 from separation unit 128 can reach a temperature of about 750° C. to about 850°° C. after heat integration with second electrolyzer 112. For example, recycled tail gas stream 134 can be passed through one or more heat exchangers (not shown) to increase its temperature to a temperature of about 750° C. to about 850° C. for heat integration with second electrolyzer 112.

In some embodiments, as depicted in FIG. 1B, second electrolyzer 112 receives fifth steam feed stream 104-2, recycled tail gas stream 134 and a methane-rich feed stream 136 containing methane and light hydrocarbons (e.g., CₓH₍₂ₓ₊₂₎ such as C₂ to C₄) into anode 112-2. In some embodiments, methane-rich feed stream 136 can be derived from a light hydrocarbon feed stream comprising methane or natural gas or renewable natural gas such as, for example, a light hydrocarbon feed stream comprising greater than about 50%, or greater than about 80%, or greater than about 90%, or greater than about 95%, or greater than about 99% methane. As used herein, natural gas or renewable natural gas comprises methane and potentially higher alkanes, carbon dioxide, nitrogen or other gases, and/or sulfur-containing compounds such as hydrogen sulfide, and mixtures thereof.

In some embodiments, as depicted in FIG. 1C, second electrolyzer 112 receives fifth steam feed stream 104-2 and methane-rich feed stream 136 into anode 112-2. FIG. 1C further shows recycled tail gas stream 134 exiting separation unit 128 where it can be further processed or reused as, for example, a fuel gas.

Recycled tail gas stream 134 and/or methane-rich feed stream 136 are fed into anode 112-2 to react with fifth steam feed stream 104-2 and oxygen migrating through electrolyte 112-3 to anode 112-2 to form anode effluent 116. In some embodiments, the methane and the other light hydrocarbon present in recycled tail gas stream 134 and/or methane-rich feed stream 136 react in anode 112-2 via a reforming reaction such as, for example, steam methane reforming, dry reforming, etc., with fifth steam feed stream 104-2 and partial oxidation with oxygen migrating through electrolyte 112-3 to anode 112-2 to produce an anode effluent 116 including at least syngas composed of mostly carbon monoxide (CO) and hydrogen (H₂) along with water (H₂O) (minimal water after a dehydration step) and minimal carbon dioxide (CO₂) having a temperature of about 750°° C. to about 850°° C. For example, in some embodiments, the light hydrocarbons in recycled tail gas stream 134 and/or methane-rich feed stream 136 could be partially oxidized into syngas as follows:

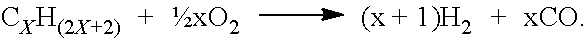

In some embodiments, methane in recycled tail gas stream 134 and/or methane-rich feed stream 136 will also serve as a fuel to provide heat and depolarize anode 112-2 and hence lower the cell voltage. Therefore, the electrical power consumption of second electrolyzer 112 can be lower than a conventional steam electrolyzer.

In an illustrative embodiment, second electrolyzer 112 can be any suitable high-temperature solid-oxide electrolyzer as discussed above for first electrolyzer 106 (See, e.g., FIG. 2).

Cathode effluent 110 (H₂-rich), cathode effluent 114 (H₂-rich) and anode effluent 116 (syngas (CO and H₂)-rich) each having a temperature of about 750° C. to about 850°° C. can be combined into a feed stream 118 and sent to one or more heat exchangers (not shown) to remove heat from cathode effluent 110, cathode effluent 114 and anode effluent 116 before being combined with a carbon dioxide feed stream 120 to form a reactor feed stream 122 composed of mainly carbon monoxide (CO), hydrogen (H₂), and minimal water (H₂O) and carbon dioxide (CO₂) and having a temperature of about 200°° C. to about 300°° C. Carbon dioxide feed stream 120 enters system 100 at or around a temperature of about 20° C. However, after undergoing heat integration with a fuel synthesis effluent stream (i.e., a reactor synthesis effluent 126), the temperature of the carbon dioxide can be increase to a range of about 200°° C. to about 300° C.

System 100 further includes a reactor unit 124 for receiving reactor feed stream 122. In some embodiments, reactor unit 124 can be one or more of a Fischer-Tropsch reactor, methanol and/or dimethyl ether reactors and a hydrogenation reactor. In some embodiments, reactor unit 124 can be a Fischer-Tropsch reactor for processing reactor feed stream 122. In some embodiments, a Fischer-Tropsch reactor is utilized for processing reactor feed stream 122 thereby converting syngas (CO and H₂) to a Fischer-Tropsch product by conventional techniques. For example, in a Fischer-Tropsch reaction, syngas composed of carbon monoxide (CO) and hydrogen gas (H₂), is converted in the presence of a Fischer-Tropsch catalyst (e.g., iron-or cobalt-based catalyst) into hydrocarbon products, water and other byproducts. For example, the Fischer-Tropsch process involves a series of chemical reactions for converting syngas to a variety of hydrocarbons, ideally having the formula (CₙH₂ₙ₊₂). In an illustrative embodiment, the more useful reactions produce alkanes such as follows:

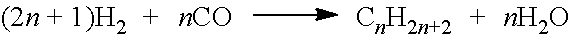

where n is from 1 to 70. In addition to the desired liquid hydrocarbon products, a Fisher-Tropsch reaction can also produce light ends (fraction of C₁ to C₄ products). In a non-limiting illustrative embodiment, Table 1 shows a product yield for Fisher-Tropsch reaction using syngas as feed.

TABLE 1

| Products | Selectivity % |
|---|---|
| Gas (C₁ to C₄) | 5 to 7 |
| Naphtha (C₅ to C₇) | 10 to 15 |
| Diesel | 30 to 35 |
| Base Oil | 45 to 50 |

The products produced from the reaction process in reactor unit 124 can then be discharged from reactor unit 124 as reactor synthesis effluent 126 comprising Fischer-Tropsch products.

In some embodiments, reactor unit 124 can be include one or more of a methanol reactor and a dimethyl ether reactor, rather than a Fischer-Tropsch reactor for processing reactor feed stream 122. For example, reactor unit 124 can be a synthesis reactor for converting CO and hydrogen in reactor feed stream 122 to such desired products as methanol which can thereafter be converted to, for example, dimethyl ether, in the same or separate reactor (not shown) by conventional techniques, e.g., by methanol synthesis and in-situ dehydration, in which the in-situ methanol conversion can alleviate the thermodynamic limits of methanol synthesis, resulting in higher dimethyl ether yield.

In a non-limiting illustrative embodiment, a methanol synthesis section can be as represented below.

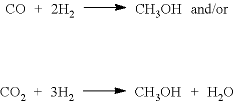

The products produced from the reaction process in reactor unit 124 can then be discharged from reactor unit 124 as reactor synthesis effluent 126 comprising one or more of methanol and/or dimethyl ether.

Numerous types of reactor systems have been developed for carrying out the Fischer-Tropsch or methanol/dimethyl ether reaction. For example, Fischer-Tropsch or methanol/dimethyl ether reactor systems include fixed bed reactors, especially multi-tubular fixed bed reactors, fluidized bed reactors, such as entrained fluidized bed reactors, and slurry bed reactors such as three-phase slurry bubble columns and ebullated bed reactors. The present invention is applicable to all types of reactor systems. The reactors each have an inlet for receiving the reactor feed stream and an outlet for discharging an effluent stream.

In this particular embodiment, system 100 can further include a reverse water gas shift (RWGS) reactor unit (not shown) for receiving reactor feed stream 122 in the case where reactor unit 124 is a Fischer-Tropsch reactor or a methanol reactor. The reverse water gas shift reaction converts CO₂ and H₂into CO and H₂O. For example, catalyst and/or reactor configurations for performing Fischer-Tropsch (FT) synthesis or methanol synthesis can readily use CO as a reactant but typically cannot use carbon dioxide (CO₂). As a result, performing the reverse water gas shift reaction can allow CO₂ and H₂ to be used to form syngas, which can then be used for a fuel synthesis reaction such as methanol synthesis.

The reverse water gas shift reaction is part of the same equilibrium as the water gas shift reaction. In that equilibrium, formation of CO and H₂O is favored by increased temperatures. In particular, due to a competing equilibrium reaction for formation of methane, the equilibrium conversion of CO₂ passes through a minimum at roughly 600° C. By performing the reverse water gas shift reaction at temperatures of 700° C. or more, or 800° C. or more, or 900°° C. or more (such as up to 1600° C. or possibly still higher), the equilibrium conversion of CO₂ can be increased while operating at temperatures with relatively fast kinetics. In various aspects, the amount of CO₂ in the reaction products can be about 0.5 vol % to about 5.0 vol %, or about 0.5 vol % to about 3.0 vol %, or about 0.5 vol % to about 2.5 vol %, or about 1.0 vol % to about 5.0 vol %, or about 1.0 vol % to about 3.0 vol %, or about 1.0 vol % to about 2.5 vol %.

The RWGS reactor unit can be a cylindrical vessel (e.g., with a length longer than diameter). The entrance to the reactor vessel can be smaller than the overall diameter of the vessel. The reactor vessel can be a steel vessel that is lined with an inert material that is non-reactive with the heated syngas. The steel vessel can be insulated to limit heat loss. Various types of insulation include poured or castable refractory lining or insulating bricks may be used to limit the heat losses to the environment.

A bed of catalyst can be inside the RWGS reactor unit. The catalyst can be in the form of granules, pellets, spheres, trilobes, quadra-lobes, monoliths, or any other engineered shape (e.g., to minimize pressure drop across the reactor). The shape and particle size of the catalyst particles can be managed such that pressure drop across the reactor is less than 50 pounds per square inch (psi) (e.g., between 10 psi and 50 psi), and in some cases, less than 20 psi (e.g., between 10 psi and 20 psi). The size of the catalyst form can have a characteristic dimension of between 1 mm to 10 mm. The catalyst particle can be a porous material with an internal surface area greater than about 80 m²/g (e.g., between about 80 m²/g and up to about 120 m²/g).

In illustrative embodiments, the RWGS catalyst is a solid solution catalyst that primarily comprises Ni₂Mg impregnated on a high-temperature spinel. This high-performance, solid-solution, Ni-based catalyst can be highly versatile and perform the RWGS reaction efficiently.

In one aspect, the RWGS reactor unit comprises one or more RWGS reactor units, arranged in series e.g., two or more RWGS reactor units. Each of the RWGS reactor units may be either adiabatic or a heated reactor. Heating can be achieved by means of heat integration with effluent of fuel synthesis reaction.

The reverse water gas shift effluent generated from reactor feed stream 122 is a stream comprising CO, H₂O, unreacted CO₂, and H₂. The reverse water gas shift effluent can have a temperature from about 400 to about 900° C., depending on the extent of reverse water gas shift reaction and extent of heating. In an illustrative embodiment, the reverse water gas shift effluent can be dehydrated before sending to reactor unit 124.

In some embodiments, reactor unit 124 can be a so-called hydrogenation reactor unit where CO₂ in reactor feed stream 122 can be directly hydrogenated into liquid hydrocarbon products such as, for example, methanol, or Fischer-Tropsch products, as known in the art. For example, in non-limiting illustrative embodiments, reactor unit 124 can be a synthesis reactor for converting carbon dioxide and hydrogen to desired products such as Fischer-Tropsch products and methanol. The methanol can thereafter be converted to, for example, dimethyl ether, in the same or separate reactor (not shown) by conventional techniques, e.g., by methanol synthesis and in-situ dehydration, in which the in-situ methanol conversion can alleviate the thermodynamic limits of methanol synthesis, resulting in higher dimethyl ether yield.

In a non-limiting illustrative embodiment, Table 2 shows a product yield for a direct hydrogenation process reaction of carbon dioxide and hydrogen.

TABLE 2

| Products | Selectivity % |
|---|---|
| Gas (C₁ to C₄) | 10 to 15 |
| Naphtha (C₅ to C₇) | 15 to 20 |
| Jet Fuel (C₈ to C₁₆) | 55 to 65 |
| Diesel (C₁₇ to C₂₃) | 5 to 10 |

The products produced from the reaction process can then be discharged from reactor unit 124 as reactor synthesis effluent 126.

Accordingly, for purposes of this illustrative embodiment, reactor synthesis effluent 126 can be one or more of Fischer-Tropsch products, one or more of methanol and/or dimethyl ether products and direct hydrogenation products as well as unconverted feeds and by-products such as methane. However, this is merely illustrative and any other product that can be made from the conversion of reactor feed stream 122 is contemplated herein for use as reactor synthesis effluent 126.

The one or more of Fischer-Tropsch products, methanol/dimethyl ether products and direct hydrogenation products can then be discharged from reactor unit 124 as reactor synthesis effluent 126 and sent to separation unit 128. The terms “separation unit” and “separator” refer to any separation device(s) that at least partially separates one or more chemical constituents in a mixture from one another. For example, a separation unit may selectively separate different chemical species from one another, forming one or more chemical fractions. Examples of separation units include, without limitation, distillation columns, fractionators, flash drums, knock-out drums, knock-out pots, centrifuges, filtration devices, traps, scrubbers, expansion devices, membranes, solvent extraction devices, high-pressure separators, low-pressure separators, and the like. It should be understood that separation processes described in this disclosure may not completely separate all of one chemical constituent from all of another chemical constituent. It should be understood that the separation processes described in this disclosure “at least partially” separate different chemical components from one another, and that even if not explicitly stated, it should be understood that separation may include only partial separation. As used in this disclosure, one or more chemical constituents may be “separated” from a process stream to form a new process stream. Generally, a process stream may enter a separation unit and be divided or separated into two or more process streams of desired composition.

The Fischer-Tropsch products, methanol/dimethyl ether products and/or direct hydrogenation products in reactor synthesis effluent 126 can be separated and collected following conventional fractional distillation to generate a product effluent 132 for sending further downstream for processing, while a portion of any unreacted syngas can be recycled to reactor unit 124 for further conversion (not shown), and any methane and light hydrocarbons such as C₁ to C₄ light ends produced from the synthesis reactions in reactor unit 124 can be recycled from separation unit 128 as recycled tail gas stream 134 back to anode 112-2 of second electrolyzer 112 (see FIGS. 1A and 1B) or sent for further processing or reused as fuel gas (see FIG. 1C) as discussed above. A liquid stream 130 including at least hydrocarbons and water is sent for further downstream processing as known in the art. For example, product effluent 132 can be sent for further downstream processing for creating high value liquid fuels, such as gasoline, diesel, and jet, or base oils.

In some embodiments, system 100 may include a hydrocracker unit and/or fractionation unit (not shown) to upgrade product effluent 126. For example, the hydrocracker unit can employ a high temperature, high pressure catalytic process that can upgrade heavy Fischer-Tropsch liquid (HFTL) and medium Fischer-Tropsch liquid (MFTL) hydrocarbon streams into a transportation fuel or a blending component meeting chemical and physical properties.

In one or more illustrative embodiments, a system processing environment 200 comprises each of the components of system 100 described herein, as well as a controller 210 operatively coupled to system 100. Controller 210 is configured to control operations of one or more of the components of system 100 discussed above. In one illustrative embodiment, controller 210 is configured to actuate one or more of the functionalities of system 100 described herein. For example, controller 210 can comprise one or more processing devices configured to load software instructions from one or more memory devices and execute the software instructions to generate data and/or control signals that can be applied to one or more components of system 100 so as to actuate the functionalities described herein. Actuation of the components by the data and/or control signals may be affected electrically, electromechanically, electrochemically, and/or the like, depending on the nature of the specific component of system 100 being actuated.

Thus, in some embodiments, controller 210 comprises a combination of hardware and software components. For example, the one or more processing devices of controller 210 may comprise one or more microprocessors, one or more microcontrollers, one or more application-specific devices, or other types of processing circuitry, as well as portions or combinations thereof. Further, the one or more memory devices of controller 210 may comprise random access memory (RAM), read-only memory (ROM), or other types of memory, in any combination. It is to be appreciated that the specific architecture of controller 210 is configurable based on the components of system 100 and the functionalities they are intended to perform.

For example, controller 210 can be operatively connected to a processing device in a processing platform which comprises a processor coupled to a memory. The processor may comprise a microprocessor, a microcontroller, an application-specific integrated circuit (ASIC), a field-programmable gate array (FPGA) or other type of processing circuitry, as well as portions or combinations of such circuitry elements. The memory may comprise random access memory (RAM), read-only memory (ROM) or other types of memory, in any combination.

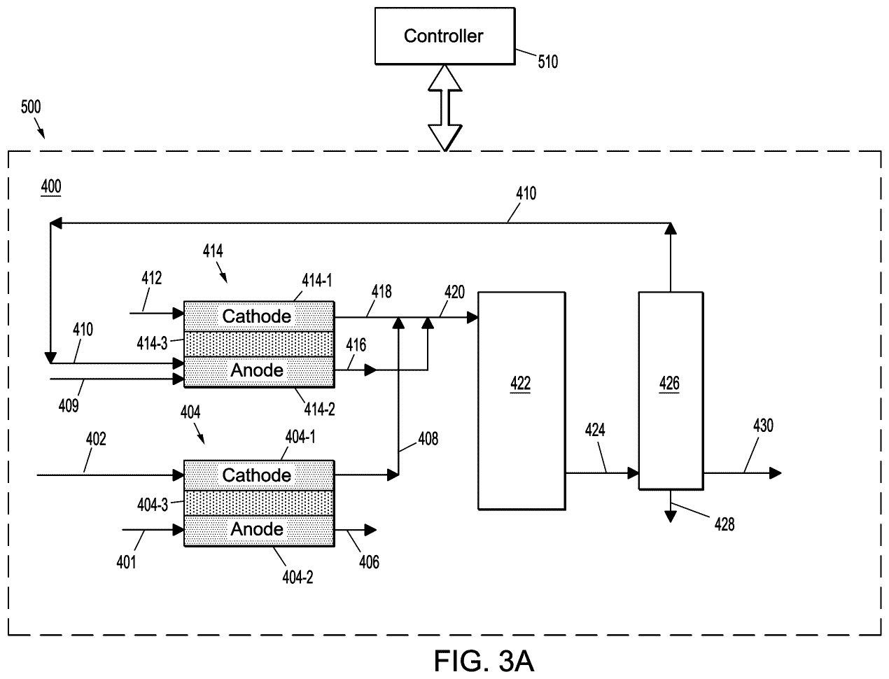

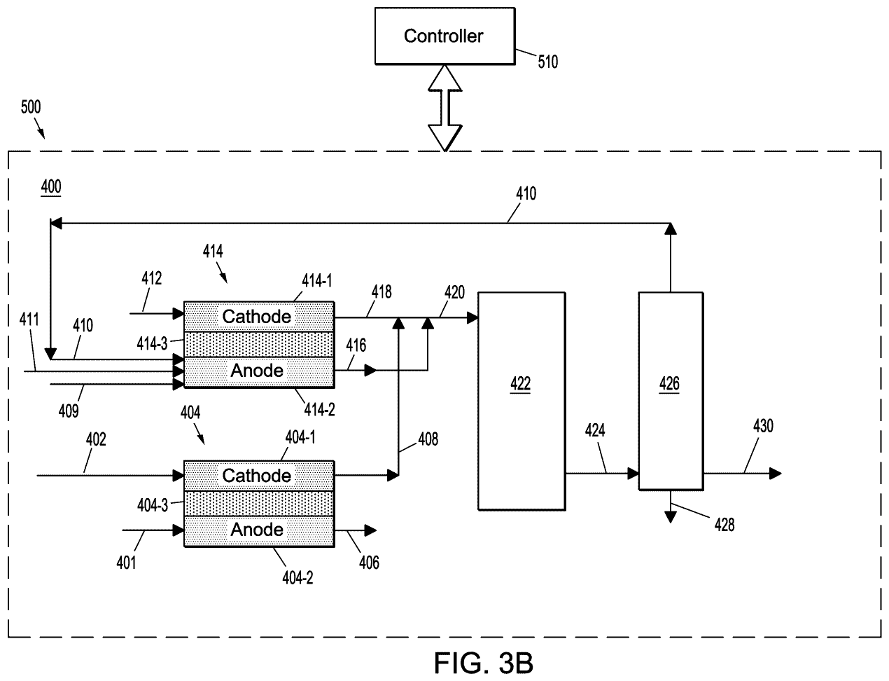

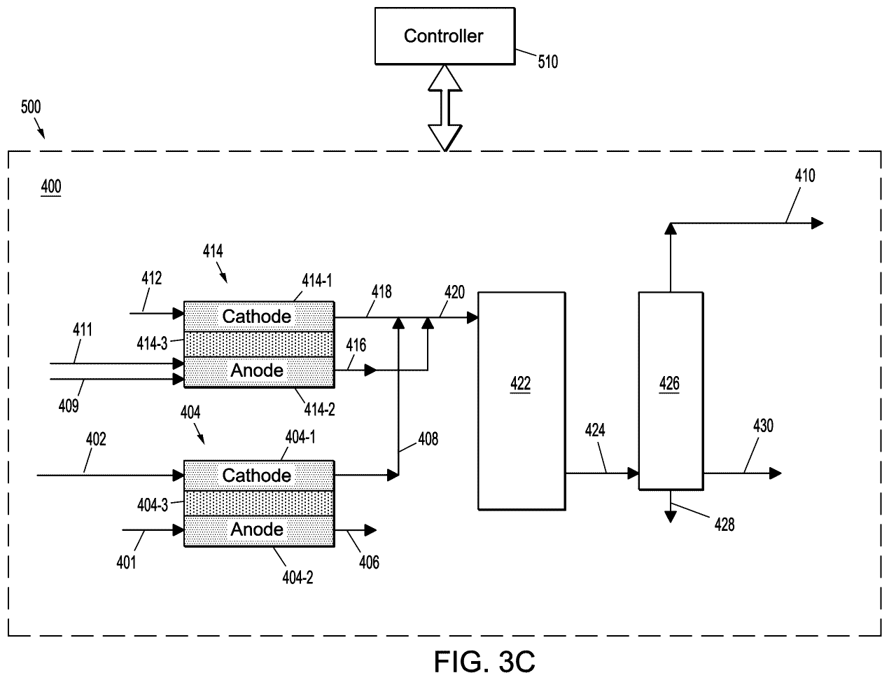

In non-limiting illustrative embodiments, a method and system for co-electrolysis of a steam and carbon dioxide feed stream to produce a mixture of carbon monoxide (CO) and hydrogen (H₂) (i.e., syngas) in a first high-temperature solid-oxide electrolyzer and co-electrolysis of a steam and carbon dioxide feed stream in the cathode to produce syngas and a reforming reaction and a partial oxidation reaction of one or more of a recycled tail gas stream and a methane-rich feed stream in the anode to produce syngas in a second high-temperature solid-oxide electrolyzer, which are then fed to a reactor unit to produce chemical products and/or fuel-based products is described with reference to FIGS. 3A-3C.

Referring now to FIGS. 3A-3C, an anode purge stream 401 and a first steam and carbon dioxide feed stream 402 are sent to a system 400. Anode purge stream 401 includes one or more of air, carbon dioxide or an inert gas such as N₂. First steam and carbon dioxide feed stream 402 can be obtained from any means known in the art. In some embodiments, anode purge stream 401 such as air may be pressurized to produce a pressurized anode purge stream as described above for anode purge stream 101.

System 400 includes a first electrolyzer 404 comprising a cathode 404-1, an anode 404-2 and an electrolyte 404-3 inserted between cathode 404-1 and anode 404-2. First electrolyzer 404 receives anode purge stream 401 into anode 404-2 and first steam and carbon dioxide feed stream 402 into cathode 404-1. First electrolyzer 404 can operate in a co-electrolysis mode in which first steam and carbon dioxide feed stream 402 participates in a reaction to produce a cathode effluent 408 including syngas composed of carbon monoxide (CO) and hydrogen (H₂) and anode purge stream 401 into anode 404-2 to produce an anode effluent 406 composed of an oxygen enriched stream from anode 404-2. Anode purge stream 401 serves as a purge gas to carry oxygen generated at anode 404-2. Anode effluent 406 can be discarded or recycled back to first electrolyzer 404.

In an illustrative embodiment, first electrolyzer 404 can be any suitable high temperature electrolyzer comprising cathode 404-1, anode 404-2 and electrolyte 404-3 inserted between anode 404-2 and cathode 404-1. In a non-limiting illustrative embodiment, first electrolyzer 404 is a high temperature solid oxide electrolyzer (also referred to as SOEC) comprising:
a first porous conductive electrode, or “cathode,” to be supplied with steam and carbon dioxide for the production of syngas composed of dihydrogen and carbon monoxide;
a second porous conductive electrode, or “anode,” via which the dioxygen (O₂) produced by the electrolysis of the water injected onto the cathode escapes; and
a solid oxide membrane (dense electrolyte) sandwiched between the cathode and the anode, the membrane being anionically conductive at high temperatures, usually temperatures above about 700°° C. and up to about 950° C.

First electrolyzer 404 may receive input energy (i.e., electricity) from an intermittent source such as solar power (including photovoltaic and reflective), wind power, tidal power, wave power, batteries, and other intermittent energy sources known in the art and combinations thereof. Alternatively, or in addition, first electrolyzer 404 may receive input energy from a non-intermittent source, such as an electricity grid (e.g., a regional electricity grid, a municipal electricity grid, or a microgrid), natural gas, coal, nuclear, and other non-intermittent sources known in the art and combinations thereof. First electrolyzer 404 may therefore be electricity connectable to an intermittent energy input, a non-intermittent source, or a combination thereof. In particular embodiments, first electrolyzer 404 may receive input energy from the photovoltaic panel.

In some embodiments, first electrolyzer 404 may be operational receiving electricity from a photovoltaic panel. At night, first electrolyzer 404 may be operated in “hot stand-by” mode to conserve electricity, or first electrolyzer 404 may be electricity connected to another power source to continue operating at night. In particular, first electrolyzer 404 may be connected to a power grid such as a regional power grid, a municipal power grid, or a micro grid, and first electrolyzer 404 may run when the price of electricity is low.

System 400 may further comprise an energy storage mechanism or a plurality of energy storage mechanisms. The energy storage mechanism may comprise any mechanism or apparatus operable to store energy such as electricity, thermal energy, etc. For example, the energy storage mechanism may include batteries (e.g., lead-acid batteries, lithium-ion batteries, lithium-ion batteries, etc.), ice, water, flywheels, compressed air, pumped hydroelectric, or other energy storage mechanisms known in the art and combinations thereof.

In illustrative embodiments, a solid oxide electrolyzer can include a stack of elementary solid-oxide (co-) electrolysis cells each comprising an anode, a cathode, and an electrolyte inserted between the anode and the cathode, the cells being electrically connected in series, the stack comprising two electrical terminals for the supply of current to the cells and defining flow chambers for, with respect to the first chambers, the flow of steam, hydrogen and carbon dioxide, and carbon monoxide over the cathodes and flow chambers for, with respect to the second chambers, the flow of air or nitrogen or oxygen or of a mixture of gases containing oxygen, and carbon dioxide over the anodes.

In illustrative embodiments, a solid oxide electrolyzer generally relies on an electron source (external source of electricity). The heat and electricity to operate the solid oxide electrolyzer may be produced from renewable sources, such as solar, wind, geothermal, or hydropower. Heat may be added to the solid oxide electrolyzer to maintain a desired operating temperature of the solid oxide electrolyzer including the electrochemical reduction. In an illustrative embodiment, heat may be added to the solid oxide electrolyzer by, for example, resistive heating (e.g., at the solid oxide electrolyzer electrodes), a steam jacket, solar heating systems, etc.

A suitable operating temperature and pressure of first electrolyzer 404 can be any of those discussed above for first electrolyzer 106.

System 400 further includes a second electrolyzer 414 comprising a cathode 414-1, an anode 414-2 and an electrolyte 414-3 inserted between cathode 414-1 and anode 414-2. Second electrolyzer 414 receives a second steam and carbon dioxide feed stream 412 into cathode 414-1 and a steam feed stream 409, and one or more of a recycled tail gas stream 410 from a separation unit 426 as discussed below and a methane-rich feed stream 411 each containing methane and light hydrocarbons (e.g., CₓH₍₂ₓ₊₂₎ such as C₂ to C₄) into anode 414-2. Second electrolyzer 414 can operate in a co-electrolysis mode in which second steam and carbon dioxide feed stream 412 participates in a reaction to produce a cathode effluent 418 including syngas composed of carbon monoxide (CO) and hydrogen (H₂) having a temperature of about 750° C. to about 850° C. with oxygen migrating through electrolyte 414-3 to anode 414-2.

In some embodiments, as depicted in FIG. 3A, second electrolyzer 414 receives steam feed stream 409 and recycled tail gas stream 410 from separation unit 426 containing methane and light hydrocarbons (e.g., CₓH₍₂ₓ₊₂₎ such as C₂ to C₄) into anode 414-2. Recycled tail gas stream 410 from separation unit 426 can reach a temperature of about 750°° C. to about 850° C. after heat integration with second electrolyzer 414. For example, recycled tail gas stream 410 can be passed through one or more heat exchangers (not shown) to increase its temperature to a temperature of about 750° C. to about 850° C. by heat integration with second electrolyzer 414.

In some embodiments, as depicted in FIG. 3B, second electrolyzer 414 receives steam feed stream 409, recycled tail gas stream 410 and methane-rich feed stream 411 each containing methane and light hydrocarbons (e.g., CₓH₍₂ₓ₊₂₎ such as C₂ to C₄) into anode 414-2. In some embodiments, methane-rich feed stream 411 can be derived from a light hydrocarbon feed stream comprising methane or natural gas such as, for example, a light hydrocarbon feed stream comprising greater than about 50%, or greater than about 80%, or greater than about 90%, or greater than about 95%, or greater than about 99% methane.

In some embodiments, as depicted in FIG. 3C, second electrolyzer 414 receives steam feed stream 409 and methane-rich feed stream 411 into anode 414-2. FIG. 3C further shows recycled tail gas stream 410 exiting separation unit 426 where it can be further processed or reused as, for example, a fuel gas.

The methane and the other light hydrocarbon present in recycled tail gas stream 410 and/or methane-rich feed stream 411 react in anode 414-2 via a reforming reaction such as, for example, steam methane reforming, dry reforming, etc., with steam feed stream 409 and partial oxidation with oxygen migrating through electrolyte 414-3 to anode 414-2 to produce an anode effluent 416 including at least syngas composed of mostly carbon monoxide (CO) and hydrogen (H₂) along with water (H₂O) (minimal water after a dehydration step) and minimal carbon dioxide (CO₂) having a temperature of about 750° C. to about 850°° C. For example, in some embodiments, the light hydrocarbons in recycled tail gas stream 410 and/or methane-rich feed stream 411 could be partially oxidized into syngas as follows:

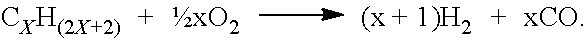

In some embodiments, methane in recycled tail gas stream 410 and/or methane-rich feed stream 411 will also serve as a fuel to provide heat and depolarize anode 414-2 and hence lower the cell voltage. Therefore, the electrical power consumption of second electrolyzer 414 can be lower than a conventional steam electrolyzer.

Second electrolyzer 414 can be any suitable high temperature electrolyzer as discussed above for second electrolyzer 112.

An advantage of the illustrative embodiments described herein is that by using co-electrolysis the composition of the syngas feed to a reactor unit 422 as discussed below can be optimized for the target synthesis reaction to achieve better performance such as higher conversion and product selectivity. For example, since syngas is produced by co-electrolysis of a steam and carbon dioxide feed stream as well as a reforming reaction of one or more of recycled tail gas stream 410 and methane-rich feed stream 411 including light hydrocarbons and partial oxidation by recycled tail gas stream 410 and/or methane-rich feed stream 411, a Fisher-Tropsch reactor can be further used to convert syngas into hydrocarbons.

Cathode effluent 408 (syngas (CO and H₂)-rich), anode effluent 416 (syngas (CO and H₂)-rich) and cathode effluent 418 (syngas (CO and H₂)-rich) each having a temperature of about 750°° C. to about 850°° C. can be sent to one or more heat exchangers (not shown) to remove heat from cathode effluent 408, anode effluent 416 and cathode effluent 418 before being combined to form a reactor feed stream 420 having a temperature of about 200°° C. to about 300° C.

System 400 further includes reactor unit 422 for receiving reactor feed stream 420. In some embodiments, reactor unit 422 can be one or more of a Fischer-Tropsch reactor, methanol and/or dimethyl ether reactors and a hydrogenation reactor. In some embodiments, reactor unit 422 can be a Fischer-Tropsch reactor for converting syngas in reactor feed stream 420 to a Fischer-Tropsch product by conventional techniques as discussed above. For example, in a Fischer-Tropsch reaction, syngas composed of carbon monoxide (CO) and hydrogen gas (H₂), is converted in the presence of a Fischer-Tropsch catalyst (e.g., iron-or cobalt-based catalyst) into hydrocarbon products, water and other byproducts. The Fischer-Tropsch reactor can be any of those discussed above. The products produced from the reaction process in reactor unit 422 can then be discharged from reactor unit 422 as a reactor synthesis effluent 424 comprising Fischer-Tropsch products.

In some embodiments, reactor unit 422 can be a methanol reactor and/or dimethyl ether reactor, rather than a Fischer-Tropsch reactor for processing reactor feed stream 420. The products produced from the reaction process in reactor unit 422 can then be discharged from reactor unit 422 as reactor synthesis effluent 424 comprising one or more of methanol and/or dimethyl ether.

In some embodiments, reactor unit 422 can be a hydrogenation reactor for direct hydrogenation of reactor feed stream 420 to produce liquid hydrocarbons as discussed above.

Accordingly, for purposes of this illustrative embodiment, reactor synthesis effluent 424 can be one or more of Fischer-Tropsch products, one or more of methanol and/or dimethyl ether products and direct hydrogenation products as well as unconverted feeds and by-products such as methane. However, this is merely illustrative and any other product that can be made from the conversion of reactor feed stream 420 is contemplated herein for use as reactor synthesis effluent 424.

The Fischer-Tropsch products, methanol/dimethyl ether products and/or direct hydrogenation products produced from the reaction process in reactor synthesis effluent 424 can then be discharged from reactor unit 422 and sent to separation unit 426. Separation unit 426 can be any of those discussed above for separation unit 128. For example, the Fischer-Tropsch products, methanol/dimethyl ether products and/or direct hydrogenation products can be separated and collected following conventional fractional distillation to produce a product effluent 430 for sending further downstream for processing, while a portion of any unreacted syngas can be recycled to reactor unit 422 for further conversion (not shown), and methane and light hydrocarbons such as C₁ to C₄ light ends produced from the synthesis reactions in reactor unit 422 can be recycled from separation unit 426 as recycled tail gas stream 410 back to anode 414-2 of second electrolyzer 414 (see FIGS. 3A and 3B) or sent for further processing or reused as, for example, a fuel gas (see FIG. 3C) as discussed above. A liquid stream 428 including at least hydrocarbons and water is sent for further downstream processing as known in the art. For example, product effluent 430 can be sent for further downstream processing for creating high value liquid fuels, such as gasoline, diesel, and jet, or base oils.

In some embodiments, system 400 may include a hydrocracker unit and/or fractionation unit to upgrade the Fischer-Tropsch liquids as discussed above for system 100.

In one or more illustrative embodiments, a system processing environment 500 comprises each of the components of system 400 described herein, as well as a controller 510 operatively coupled to system processing environment 500. Controller 510 is configured to control operations of one or more of the components of system 400 discussed above. In one illustrative embodiment, controller 510 is configured to actuate one or more of the functionalities of system 400 described herein. For example, controller 510 can be similar to controller 210 described above and function in a similar manner.

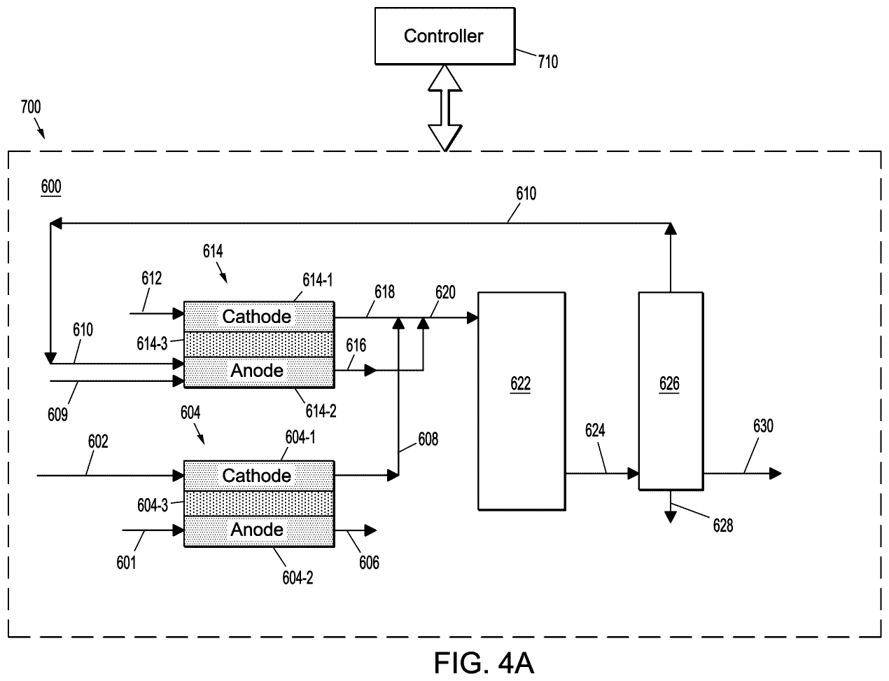

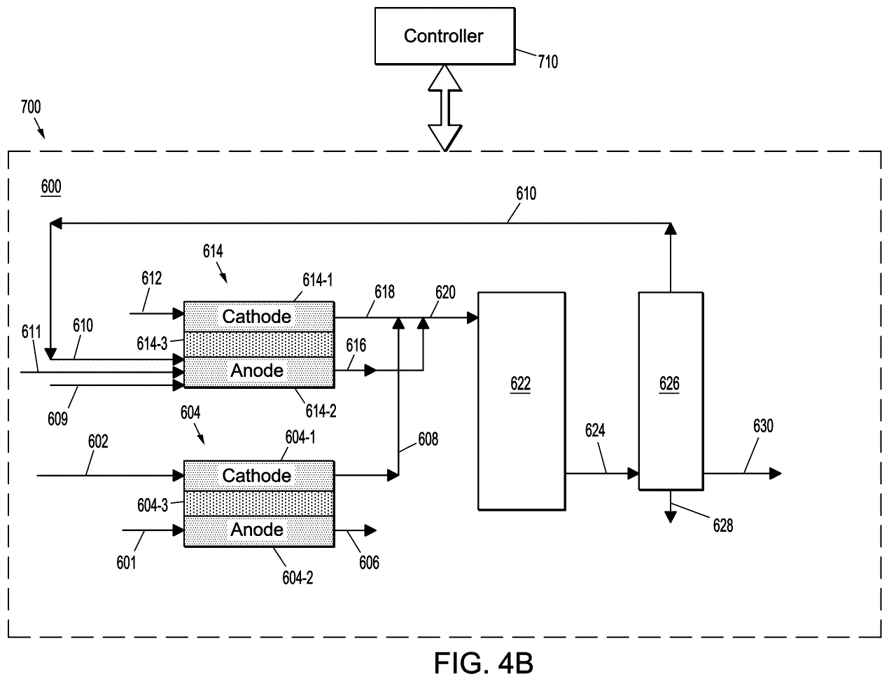

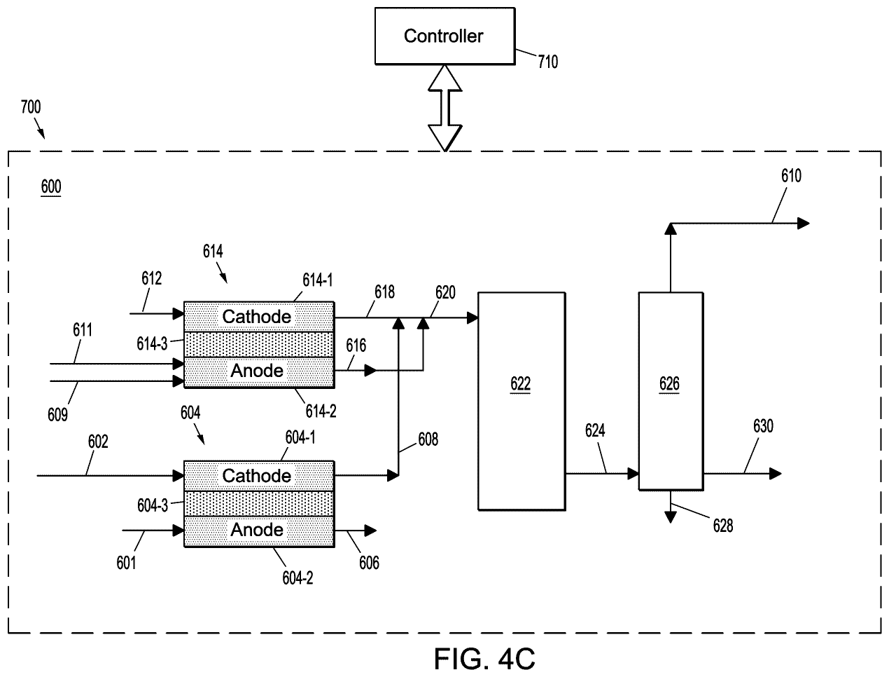

In non-limiting illustrative embodiments, a method and system for co-electrolysis of a steam and carbon dioxide feed stream to produce a mixture of carbon monoxide (CO) and hydrogen (H₂) (i.e., syngas) in a first high-temperature solid-oxide electrolyzer and steam electrolysis of a steam feed stream in the cathode to produce hydrogen and a reforming reaction and a partial oxidation reaction of one or more of a recycled tail gas stream and a methane-rich feed stream in the anode to produce at least syngas in a second high-temperature solid-oxide electrolyzer, which are then fed to a reactor unit to produce chemical products and/or fuel-based products is described with reference to FIGS. 4A-4C.

Referring now to FIGS. 4A-4C, an anode purge stream 601 and a steam and carbon dioxide feed stream 602 are sent to a system 600. Anode purge stream 601 includes one or more of air, carbon dioxide or an inert gas such as N₂. Steam and carbon dioxide feed stream 602 can be obtained from any means known in the art. In some embodiments, anode purge stream 601 such as air may be pressurized to produce a pressurized anode purge stream as described above for anode purge stream 101.

System 600 includes a first electrolyzer 604 comprising a cathode 604-1, an anode 604-2 and an electrolyte 604-3 inserted between cathode 604-1 and anode 604-2. First electrolyzer 604 receives anode purge stream 601 into anode 604-2 and steam and carbon dioxide feed stream 602 into cathode 604-1. First electrolyzer 604 can operate in a co-electrolysis mode in which steam and carbon dioxide feed stream 602 participates in a reaction to produce a cathode effluent 608 including syngas composed of carbon monoxide (CO) and hydrogen (H₂) and anode purge stream 601 into anode 604-2 to produce an anode effluent 606 composed of an oxygen enriched stream from anode 604-2. Anode purge stream 601 serves as a purge gas to carry oxygen generated at anode 604-2. Anode effluent 606 can be discarded or recycled back to first electrolyzer 604.

First electrolyzer 604 can be any suitable high temperature electrolyzer as discussed above for first electrolyzer 404.

System 600 further includes a second electrolyzer 614 comprising a cathode 614-1, an anode 614-2 and an electrolyte 614-3 inserted between cathode 614-1 and anode 614-2. Second electrolyzer 614 receives steam feed stream 612 into cathode 614-1 and a steam feed stream 609, and one or more of a recycled tail gas stream 610 from a separation unit 626 as discussed below and/or a methane-rich feed stream 611 each containing methane and light hydrocarbons (e.g., CₓH₍₂ₓ₊₂₎ such as C₂ to C₄) into anode 614-2. Second electrolyzer 614 can operate in an electrolysis mode in which steam feed stream 612 is converted to a cathode effluent 618 composed mainly of H₂having a temperature of about 750° C. to about 850° C. with oxygen migrating through electrolyte 614-3 to anode 614-2.

In some embodiments, as depicted in FIG. 4A, second electrolyzer 614 receives steam feed stream 609 and recycled tail gas stream 610 from separation unit 626 containing methane and light hydrocarbons (e.g., CₓH₍₂ₓ₊₂₎ such as C₂ to C₄) into anode 614-2. Recycled tail gas stream 610 from separation unit 626 can reach a temperature of about 750°° C. to about 850° C. after heat integration with second electrolyzer 614. For example, recycled tail gas stream 610 can be passed through one or more heat exchangers (not shown) to increase its temperature to a temperature of about 750°° C. to about 850° C. for heat integration with second electrolyzer 614.

In some embodiments, as depicted in FIG. 4B, second electrolyzer 614 receives steam feed stream 609, recycled tail gas stream 610 and methane-rich feed stream 611 containing methane and light hydrocarbons (e.g., CₓH₍₂ₓ₊₂₎ such as C₂ to C₄) into anode 614-2. In some embodiments, methane-rich feed stream 611 can be derived from a light hydrocarbon feed stream comprising methane or natural gas such as, for example, a light hydrocarbon feed stream comprising greater than about 50%, or greater than about 80%, or greater than about 90%, or greater than about 95%, or greater than about 99% methane.

In some embodiments, as depicted in FIG. 4C, second electrolyzer 614 receives steam feed stream 609 and methane-rich feed stream 611 into anode 614-2. FIG. 4C further shows recycled tail gas stream 610 exiting separation unit 626 where it can be sent for further processing or reused as, for example, a fuel gas.

The methane and the other light hydrocarbon present in recycled tail gas stream 610 and/or methane-rich feed stream 611 react in anode 614-2 via a reforming reaction such as, for example, steam methane reforming, dry reforming, etc., with steam feed stream 609 and partial oxidation with oxygen migrating through electrolyte 614-3 to anode 614-2 to produce an anode effluent 616 including at least syngas composed of mostly carbon monoxide (CO) and hydrogen (H₂) along with water (H₂O) (minimal water after a dehydration step) and minimal carbon dioxide (CO₂) having a temperature of about 750°° C. to about 850° C. For example, in some embodiments, the light hydrocarbons in recycled tail gas stream 610 and/or methane-rich feed stream 611 could be partially oxidized into syngas as follows:

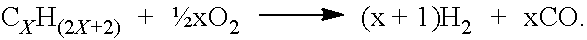

In some embodiments, methane in recycled tail gas stream 610 and/or methane-rich feed stream 611 will also serve as a fuel to provide heat and depolarize anode 614-2 and hence lower the cell voltage. Therefore, the electrical power consumption of second electrolyzer 614 can be lower than a conventional steam electrolyzer.

Second electrolyzer 614 can be any suitable high temperature electrolyzer as discussed above for first electrolyzer 106).

Cathode effluent 608 (syngas (CO and H₂)-rich), anode effluent 616 (syngas (CO and H₂)-rich) and cathode effluent 618 (H₂-rich) each having a temperature of about 750° C. to about 850°° C. can be sent to one or more heat exchangers (not shown) to remove heat from cathode effluent 608, anode effluent 616 and cathode effluent 618 before being combined to form a reactor feed stream 620 having a temperature of about 200°° C. to about 300° C.

System 600 further includes a reactor unit 622 for receiving reactor feed stream 620. In some embodiments, reactor unit 622 can be one or more of a Fischer-Tropsch reactor, methanol and/or dimethyl ether reactors and a hydrogenation reactor. In some embodiments, reactor unit 622 can be a Fischer-Tropsch reactor for converting syngas in reactor feed stream 620 to a Fischer-Tropsch product by conventional techniques as discussed above. For example, in a Fischer-Tropsch reaction, syngas composed of carbon monoxide (CO) and hydrogen gas (H₂), is converted in the presence of a Fischer-Tropsch catalyst (e.g., iron-or cobalt-based catalyst) into hydrocarbon products, water and other byproducts. The Fischer-Tropsch reactor can be any of those discussed above. The products produced from the reaction process in reactor unit 622 can then be discharged from reactor unit 622 as a reactor synthesis effluent 624 comprising Fischer-Tropsch products.

In some embodiments, reactor unit 622 can be a methanol reactor and/or dimethyl ether reactor, rather than a Fischer-Tropsch reactor for processing reactor feed stream 620. The products produced from the reaction process in reactor unit 622 can then be discharged from reactor unit 622 as reactor synthesis effluent 624 comprising one or more of methanol and/or dimethyl ether.

In some embodiments, reactor unit 622 can be a hydrogenation reactor for the direct hydrogenation of reactor feed stream 620 to produce liquid hydrocarbons as discussed above.

Accordingly, for purposes of this illustrative embodiment, reactor synthesis effluent 624 can be one or more of Fischer-Tropsch products, one or more of methanol and/or dimethyl ether products and direct hydrogenation products as well as unconverted feeds and by-products such as methane. However, this is merely illustrative and any other product that can be made from the conversion of reactor feed stream 620 is contemplated herein for use as reactor synthesis effluent 624.

The Fischer-Tropsch products, methanol/dimethyl ether products and/or direct hydrogenation products produced from the reaction process as reactor synthesis effluent 624 can then be discharged from reactor unit 622 and sent to separation unit 626. Separation unit 626 can be any of those discussed above for separation unit 128. For example, the Fischer-Tropsch products, methanol/dimethyl ether products and/or direct hydrogenation products can be separated and collected following conventional fractional distillation to produce a product effluent 630 for sending further downstream for processing, while a portion of any unreacted syngas can be recycled to reactor unit 622 for further conversion (not shown), and any methane and light hydrocarbons such as C₁ to C₄ light ends produced from the synthesis reactions in reactor unit 622 can be recycled from separation unit 626 as recycled tail gas stream 610 back to anode 614-2 of second electrolyzer 614 (see FIGS. 4A and 4B) or sent for further processing or reused as, for example, a fuel gas (see FIG. 4C) as discussed above. A liquid stream 628 including at least hydrocarbons and water is sent for further downstream processing as known in the art. For example, product effluent 630 can be sent for further downstream processing for creating high value liquid fuels, such as gasoline, diesel, and jet, or base oils.

In some embodiments, system 600 may include a hydrocracker unit and/or fractionation unit to upgrade the Fischer-Tropsch liquids as discussed above for system 100.

In one or more illustrative embodiments, a system processing environment 700 comprises each of the components of system 600 described herein, as well as a controller 710 operatively coupled to system processing environment 700. Controller 710 is configured to control operations of one or more of the components of system 600 discussed above. In one illustrative embodiment, controller 710 is configured to actuate one or more of the functionalities of system 600 described herein. For example, controller 710 can be similar to controller 210 described above and function in a similar manner.

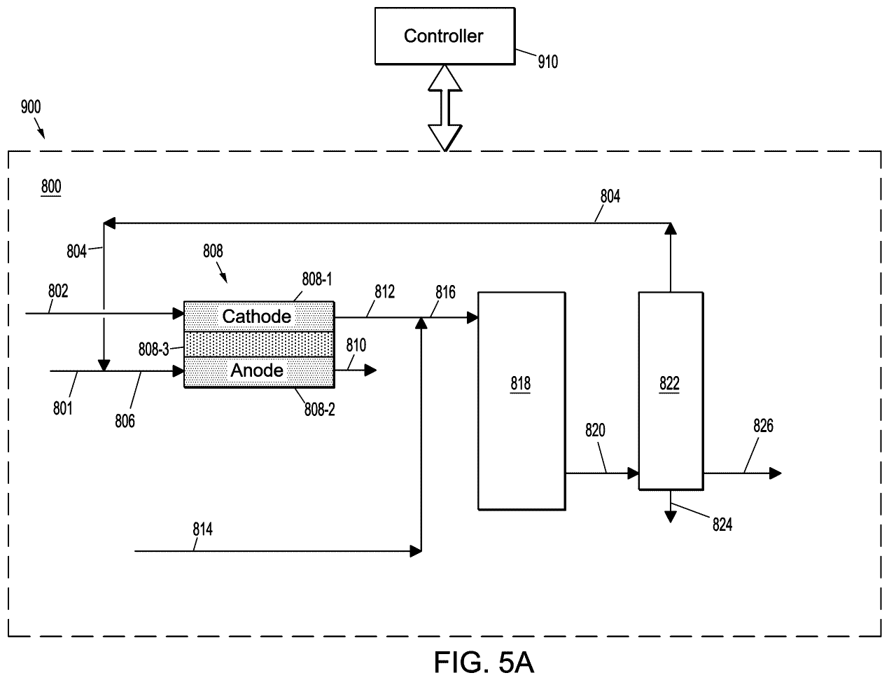

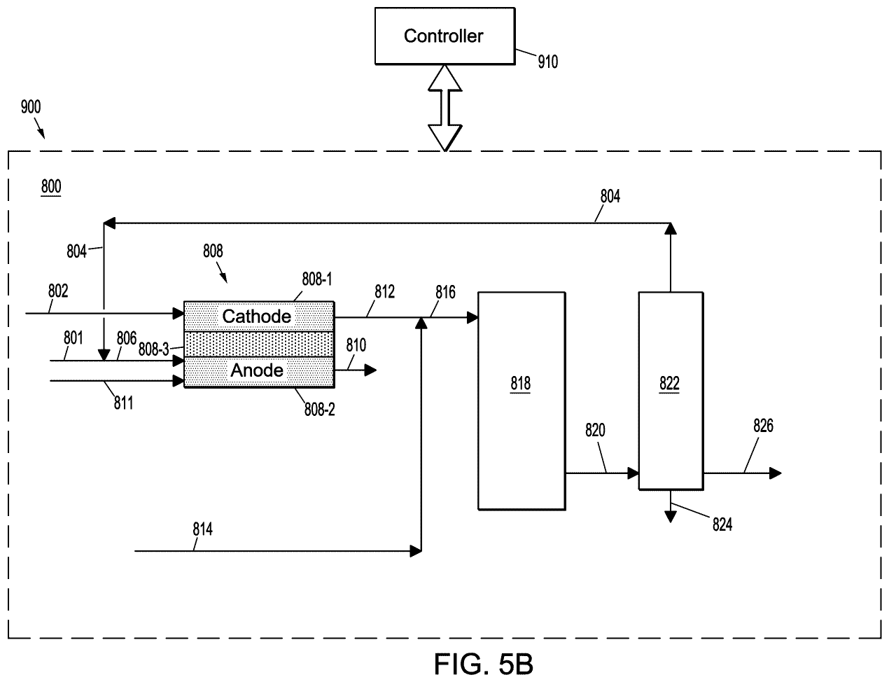

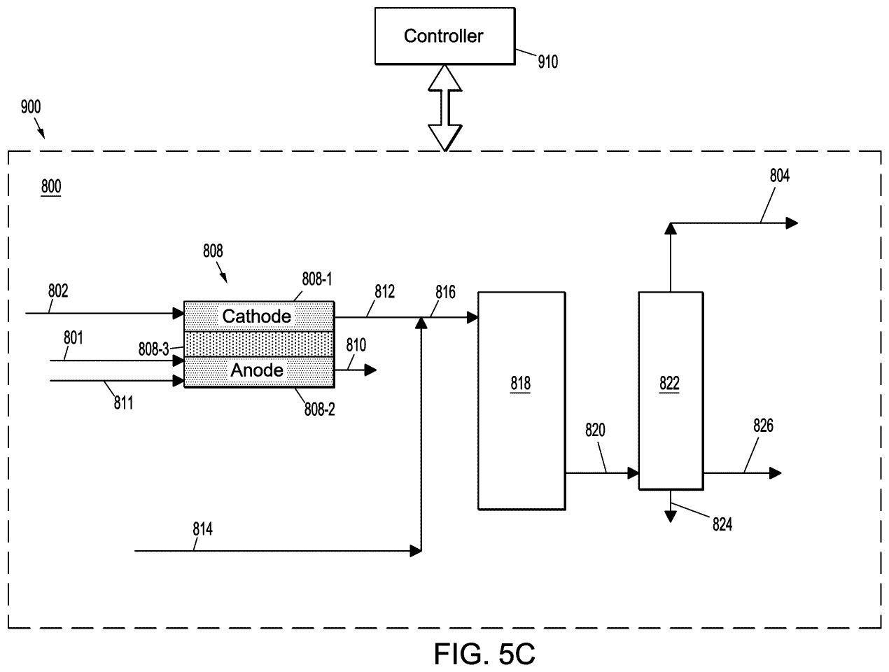

In non-limiting illustrative embodiments, a method and system for steam electrolysis of a steam feed stream in the cathode to produce hydrogen and using a recycled tail gas stream and/or a methane-rich feed stream as a fuel in the anode in a high-temperature solid-oxide electrolyzer for combining the hydrogen with a carbon dioxide feed, which are then fed to a reactor unit to produce chemical products and/or fuel-based products is described with reference to FIGS. 5A-5C.

Referring now to FIGS. 5A-5C, a system 800 includes an electrolyzer 808 comprising a cathode 808-1, an anode 808-2 and an electrolyte 808-3 inserted between cathode 808-1 and anode 808-2. Electrolyzer 808 receives a steam feed stream 802 into cathode 808-1 of electrolyzer 808 and an anode feed stream 806 formed from an anode purge stream 801, and a recycled tail gas stream 804 from a separation unit 822 as discussed below and/or methane-rich feed stream 811 into anode 808-2 of electrolyzer 808. Anode purge stream 801 includes one or more of air, carbon dioxide or an inert gas such as N₂. In some embodiments, anode purge stream 801 such as air may be pressurized to produce a pressurized anode purge stream as described above for anode purge stream 101.

In some embodiments, as depicted in FIG. 5A, electrolyzer 808 receives anode feed stream 806 formed from anode purge stream 801 and recycled tail gas stream 804 containing methane and light hydrocarbons (e.g., CₓH₍₂ₓ₊₂₎ such as C₂ to C₄) into anode 808-2. Recycled tail gas stream 804 from separation unit 822 can reach a temperature of about 750° C. to about 850°° C. after heat integration with electrolyzer 808. For example, recycled tail gas stream 804 can be passed through one or more heat exchangers (not shown) to increase its temperature to a temperature of about 750°° C. to about 850°° C. for heat integration with electrolyzer 808.

In some embodiments, as depicted in FIG. 5B, electrolyzer 808 receives anode feed stream 806 formed from anode purge stream 801 and recycled tail gas stream 804, and a methane-rich feed stream 811 containing methane and light hydrocarbons (e.g., CₓH₍₂ₓ₊₂₎ such as C₂ to C₄) into anode 808-2. In some embodiments, methane-rich feed stream 811 can be derived from a light hydrocarbon feed stream comprising methane or natural gas such as, for example, a light hydrocarbon feed stream comprising greater than about 50%, or greater than about 80%, or greater than about 90%, or greater than about 95%, or greater than about 99% methane.

In some embodiments, as depicted in FIG. 5C, electrolyzer 808 receives anode purge stream 801 and methane-rich feed stream 811 into anode 808-2. FIG. 5C further shows recycled tail gas stream 804 exiting separation unit 822 where it can be further processed or reused as, for example, a fuel gas.

Steam feed stream 802 can be converted in cathode 808-1 to a cathode effluent 812 (H₂-rich) having a temperature of about 750°° C. to about 850°° C. from cathode 808-1 with oxygen migrating through electrolyte 808-3 to anode 808-2 to produce an anode effluent 810. Recycled tail gas stream 804 and/or methane-rich feed stream 811 serve as a fuel to provide heat and depolarize anode 808-2 and hence lower the cell voltage. Therefore, the electrical power consumption of electrolyzer 808 can be lower than a conventional steam electrolyzer. Anode effluent 810 can contain oxidation products such as O₂, CO₂, N₂, H₂, CO etc., as well as water (H₂O) from oxidation and steam reforming of recycled tail gas stream 804. Due to the excessive oxygen present in anode 808-2, the concentrations of partial oxidation products such as CO and H₂will be minimal. Thus, the H₂O and CO₂ from methane oxidation at anode 808-2 can be utilized. For example, in some embodiments, the light hydrocarbons in recycled tail gas stream 804 and/or methane-rich feed stream 811 could be partially oxidized into syngas as follows:

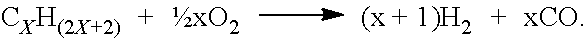

Electrolyzer 808 can be any suitable high temperature electrolyzer as discussed above for first electrolyzer 106.

Cathode effluent 812 (H₂-rich) having a temperature of about 750° C. to about 850°° C. can be sent to one or more heat exchangers (not shown) to remove heat from cathode effluent 812 before being combined with a carbon dioxide feed stream 814 to form a reactor feed stream 816 having a temperature of about 200°° C. to about 300°° C.

System 800 further includes a reactor unit 818 for receiving reactor feed stream 816. In some embodiments, reactor unit 818 can be one or more of a Fischer-Tropsch reactor, methanol and/or dimethyl ether reactors and a hydrogenation reactor. In some embodiments, reactor unit 818 can be a Fischer-Tropsch reactor for converting syngas from a RWGS reaction of reactor feed stream 816 to a Fischer-Tropsch product by conventional techniques as discussed above. For example, in a Fischer-Tropsch reaction, syngas composed of carbon monoxide (CO) and hydrogen gas (H₂), is converted in the presence of a Fischer-Tropsch catalyst (e.g., iron-or cobalt-based catalyst) into hydrocarbon products, water and other byproducts. The Fischer-Tropsch reactor can be any of those discussed above. The products produced from the reaction process in reactor unit 818 can then be discharged from reactor unit 818 as a reactor synthesis effluent 820 comprising Fischer-Tropsch products.

In some embodiments, reactor unit 818 can be a methanol reactor and/or dimethyl ether reactor, rather than a Fischer-Tropsch reactor for processing reactor feed stream 816. The products produced from the reaction process in reactor unit 818 can then be discharged from reactor unit 818 as reactor synthesis effluent 820 comprising one or more of methanol and/or dimethyl ether.

In some embodiments, reactor unit 818 can be a hydrogenation reactor for the direct hydrogenation of reactor feed stream 816 to produce liquid hydrocarbons as discussed above.

Accordingly, for purposes of this illustrative embodiment, reactor synthesis effluent 820 can be one or more of Fischer-Tropsch products, one or more of methanol and/or dimethyl ether products and direct hydrogenation products as well as unconverted feeds and by-products such as methane. However, this is merely illustrative and any other product that can be made from the conversion of reactor feed stream 816 is contemplated herein for use as reactor synthesis effluent 820.

The Fischer-Tropsch products, methanol/dimethyl ether products and/or direct hydrogenation products produced from the reaction process as reactor synthesis effluent 820 can then be discharged from reactor unit 818 and sent to separation unit 822. Separation unit 822 can be any of those discussed above for separation unit 128. For example, the Fischer-Tropsch products, methanol/dimethyl ether products and/or direct hydrogenation products can be separated and collected following conventional fractional distillation to produce a product effluent 826 for sending further downstream for processing, while a portion of any unreacted syngas can be recycled back to reactor unit 818 for further conversion (not shown), and any methane and light hydrocarbons such as C₁ to C₄ light ends produced from the synthesis reactions in reactor unit 818 can be recycled from separation unit 822 as recycled tail gas stream 804 back to anode 808-2 of electrolyzer 808 (see FIGS. 5A and 5B) or sent for further processing or reused as, for example, a fuel gas (see FIG. 5C) as discussed above. A liquid stream 824 including at least hydrocarbons and water is sent for further downstream processing as known in the art. For example, product effluent 826 can be sent for further downstream processing for creating high value liquid fuels, such as gasoline, diesel, and jet, or base oils.

In some embodiments, system 800 may include a hydrocracker unit and/or fractionation unit to upgrade the Fischer-Tropsch liquids as discussed above for system 100.

In one or more illustrative embodiments, a system processing environment 900 comprises each of the components of system 800 described herein, as well as a controller 910 operatively coupled to system processing environment 900. Controller 910 is configured to control operations of one or more of the components of system 800 discussed above. In one illustrative embodiment, controller 910 is configured to actuate one or more of the functionalities of system 800 described herein. For example, controller 910 can be similar to controller 210 described above and function in a similar manner.

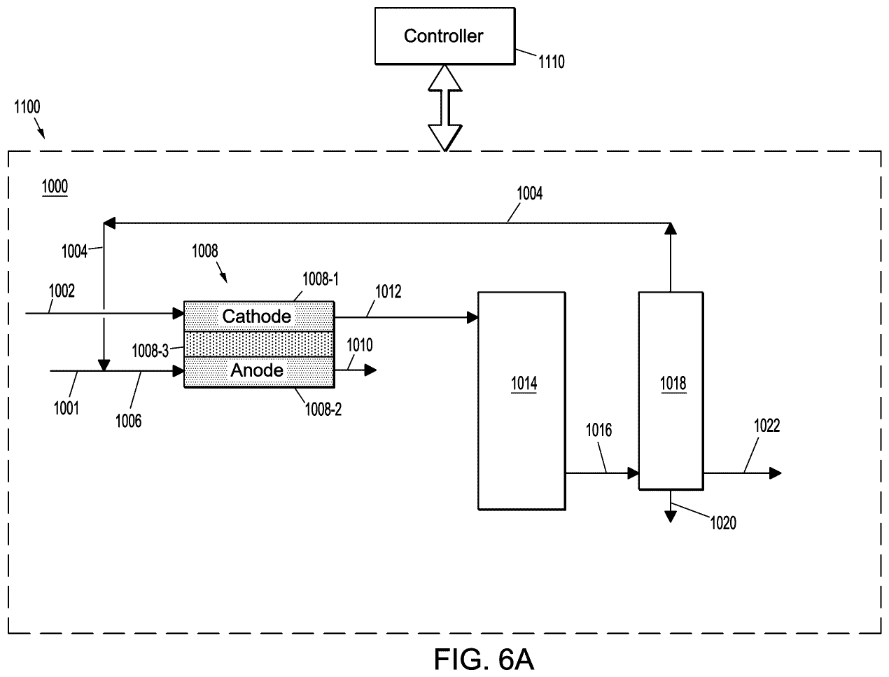

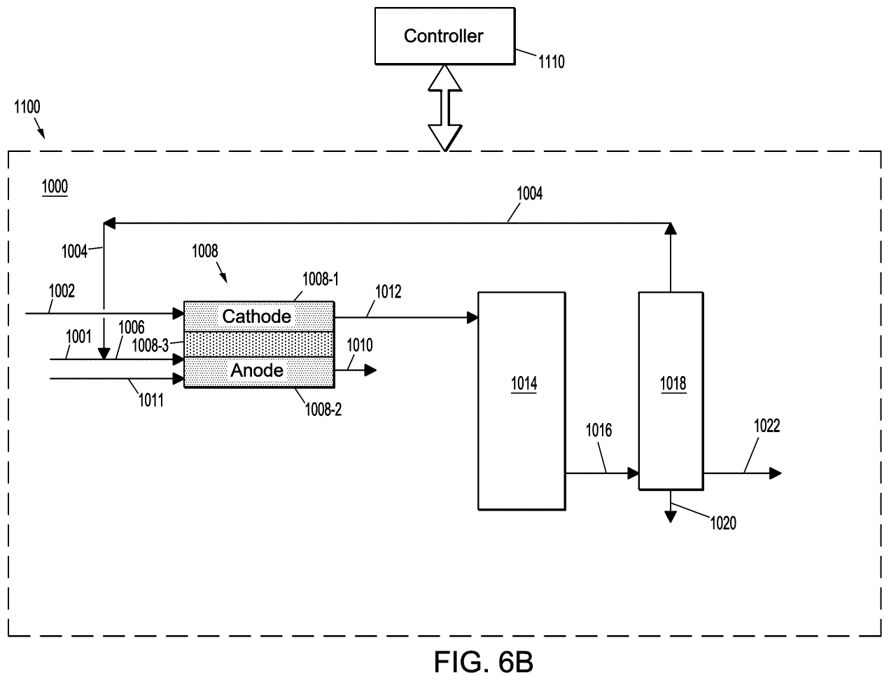

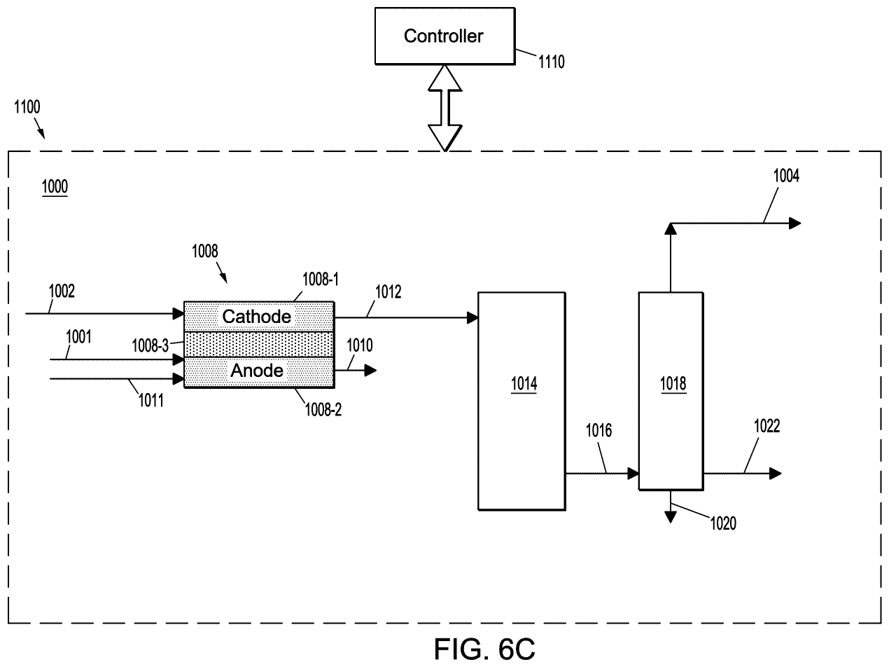

In non-limiting illustrative embodiments, a method and system for co-electrolysis of a steam and carbon dioxide feed stream in the cathode to produce syngas in a high-temperature solid-oxide electrolyzer using methane and light hydrocarbons from a recycled tail gas stream or/and methane-rich feed stream in the anode as fuel, which is then fed to a reactor unit to produce chemical products and/or fuel-based products is described with reference to FIGS. 6A-6C.

Referring now to FIGS. 6A-6C, a system 1000 includes an electrolyzer 1008 comprising a cathode 1008-1, an anode 1008-2 and an electrolyte 1008-3 inserted between cathode 1008-1 and anode 1008-2. Electrolyzer 1008 receives a steam and carbon dioxide feed stream 1002 into cathode 1008-1 and an anode feed stream 1006 formed from an anode purge stream 1001, and a recycled tail gas stream 1004 from a separation unit 1018 as discussed below and/or methane-rich feed stream 1011 into anode 1008-2 of electrolyzer 1008. Anode purge stream 1001 includes one or more of air, carbon dioxide or an inert gas such as N₂. In some embodiments, anode purge stream 1001 such as air may be pressurized to produce a pressurized anode purge stream as described above for anode purge stream 101.

In some embodiments, as depicted in FIG. 6A, electrolyzer 1008 receives anode feed stream 1006 formed from anode purge stream 1001 and recycled tail gas stream 1004 containing methane and light hydrocarbons (e.g., CₓH₍₂ₓ₊₂₎ such as C₂ to C₄) into anode 1008-2. Recycled tail gas stream 1004 from separation unit 1018 can reach a temperature of about 750° C. to about 850° C. after heat integration with electrolyzer 1008. For example, recycled tail gas stream 1004 can be passed through one or more heat exchangers (not shown) to increase its temperature to a temperature of about 750°° C. to about 850°° C. for heat integration with electrolyzer 1008.

In some embodiments, as depicted in FIG. 6B, electrolyzer 1008 receives anode feed stream 1006 formed from anode purge stream 1001 and recycled tail gas stream 1004, and methane-rich feed stream 1011 containing methane and light hydrocarbons (e.g., CₓH₍₂ₓ₊₂₎ such as C₂ to C₄) into anode 1008-2. In some embodiments, methane-rich feed stream 1011 can be derived from a light hydrocarbon feed stream comprising methane or natural gas such as, for example, a light hydrocarbon feed stream comprising greater than about 50%, or greater than about 80%, or greater than about 90%, or greater than about 95%, or greater than about 99% methane.

In some embodiments, as depicted in FIG. 6C, electrolyzer 1008 receives anode purge stream 1001 and methane-rich feed stream 1011 into anode 1008-2. FIG. 6C further shows recycled tail gas stream 1004 exiting separation unit 1018 where it can be further processed or reused as, for example, a fuel gas.

Electrolyzer 1008 can operate in a co-electrolysis mode in which steam and carbon dioxide feed stream 1002 participates in a reaction to generate a cathode effluent 1012 including syngas composed of carbon monoxide (CO) and hydrogen (H₂) having a temperature of about 750°° C. to about 850° C. from cathode 1008-1 with oxygen migrating through electrolyte 1008-3 to anode 1008-2 to produce an anode effluent 1010. Recycled tail gas stream 1004 and/or methane-rich feed stream 1011 will serve as a fuel to provide heat and depolarize anode 1008-2 and hence lower the cell voltage. Therefore, the electrical power consumption of electrolyzer 1008 can be lower than a conventional steam electrolyzer. Anode effluent 1010 can contain oxidation products such as O₂, CO₂, N₂, H₂, etc., as well as water (H₂O) from oxidation and reforming of recycled tail gas stream 1004. Due to the excessive oxygen present in anode 1008-2, the concentrations of partial oxidation and reforming products such as CO and H₂will be minimal. Thus, the H₂O and CO₂ from the tail gas oxidation and reforming at anode 1008-2 can be utilized. For example, in some embodiments, the light hydrocarbons in recycled tail gas stream 1004 and/or methane-rich feed stream 1011 could be partially oxidized into syngas as follows:

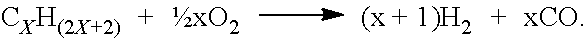

Electrolyzer 1008 can be any suitable high temperature electrolyzer as discussed above for first electrolyzer 404.

System 1000 further includes a reactor unit 1014 for receiving cathode effluent 1012 including syngas. In some embodiments, cathode effluent 1012 can be sent to one or more heat exchangers (not shown) to remove heat from cathode effluent 1012 thereby having a temperature of about 200°° C. to about 300°° C.

In some embodiments, reactor unit 1014 can be one or more of a Fischer-Tropsch reactor, methanol and/or dimethyl ether reactors and a hydrogenation reactor. In some embodiments, reactor unit 1014 can be a Fischer-Tropsch reactor for converting syngas in cathode effluent 1012 to a Fischer-Tropsch product by conventional techniques as discussed above. For example, in a Fischer-Tropsch reaction, syngas composed of carbon monoxide (CO) and hydrogen gas (H₂), is converted in the presence of a Fischer-Tropsch catalyst (e.g., iron-or cobalt-based catalyst) into hydrocarbon products, water and other byproducts. The Fischer-Tropsch reactor can be any of those discussed above. The products produced from the reaction process in reactor unit 1014 can then be discharged from reactor unit 1014 as a reactor synthesis effluent 1016 comprising Fischer-Tropsch products.

In some embodiments, reactor unit 1014 can be a methanol reactor and/or dimethyl ether reactor, rather than a Fischer-Tropsch reactor for processing cathode effluent 1012. The products produced from the reaction process in reactor unit 1014 can then be discharged from reactor unit 1014 as reactor synthesis effluent 1016 comprising one or more of methanol and/or dimethyl ether.

In some embodiments, reactor unit 1014 can be a hydrogenation reactor for the direct hydrogenation of cathode effluent 1012 to produce liquid hydrocarbons as discussed above.

Accordingly, for purposes of this illustrative embodiment, reactor synthesis effluent 1016 can be one or more of Fischer-Tropsch products, one or more of methanol and/or dimethyl ether products and direct hydrogenation products as well as unconverted feeds and by-products such as methane. However, this is merely illustrative and any other product that can be made from the conversion of cathode effluent 1012 is contemplated herein for use as reactor synthesis effluent 1016.

The Fischer-Tropsch products, methanol/dimethyl ether products and/or direct hydrogenation products produced from the reaction process as reactor synthesis effluent 1016 can then be discharged from reactor unit 1014 and sent to separation unit 1018. Separation unit 1018 can be any of those discussed above for separation unit 128. For example, the Fischer-Tropsch products, methanol/dimethyl ether products and/or direct hydrogenation products can be separated and collected following conventional fractional distillation to produce a product effluent 1022 for sending further downstream for processing, while a portion of any unreacted syngas can be recycled to reactor unit 1014 for further conversion (not shown), and any methane and light hydrocarbons such as C₁ to C₄ light ends produced from the synthesis reactions in reactor unit 1014 can be recycled from separation unit 1018 as recycled tail gas stream 1004 back to anode 1008-2 of electrolyzer 1008 (see FIGS. 6A and 6B) or sent for further processing or reused as fuel gas (sec FIG. 6C) as discussed above. A liquid stream 1020 including at least hydrocarbons and water is sent for further downstream processing as known in the art. For example, product effluent 1022 can be sent for further downstream processing for creating high value liquid fuels, such as gasoline, diesel, and jet, or base oils.

In some embodiments, system 1000 may include a hydrocracker unit and/or fractionation unit to upgrade the Fischer-Tropsch liquids as discussed above for system 100.

In one or more illustrative embodiments, a system processing environment 1100 comprises each of the components of system 1000 described herein, as well as a controller 1110 operatively coupled to system processing environment 1100. Controller 1110 is configured to control operations of one or more of the components of system 1000 discussed above. In one illustrative embodiment, controller 1110 is configured to actuate one or more of the functionalities of system 1000 described herein. For example, controller 1110 can be similar to controller 210 described above and function in a similar manner.

The following illustrative examples are intended to be non-limiting.

### EXAMPLE 1

The improvement of the energy efficiency obtained by recycling a CH₄ stream together with a steam feed to the anode of a steam electrolyzer, where steam methane reforming, partial oxidation, CO and H₂ oxidation, and WGS takes place, was evaluated via process simulations. Example 1 is illustrative of the method and system of FIG. 3A. The key performance indicators are summarized in Table 3.

TABLE 3

| | Recycle C₁ | Recycle C₁ | No C₁ |
|---|---|---|---|
| | in Anode | in Cathode | to SOEC |
| Total H₂O feed | 5071 | 5177 | 6300 |
| (kmol/hr) to First | | | |
| Electrolyzer 404, and | | | |
| Second Electrolyzer | | | |
| 414 | | | |
| CO₂ feed (kmol/hr) to | 1609 | 1609 | 2114 |
| First Electrolyzer 404 | | | |
| CH₄ feed (kmol/hr) to | 536 | 536 | N/A |
| Second Electrolyzer | | | |
| 414 | | | |
| H₂O conv. in SOEC in | 80% | 89% | 82% |
| First Electrolyzer 404, | | | |
| and Second | | | |
| Electrolyzer 414 | | | |
| CO₂ conv. in SOEC in | 74% | 81% | 78% |
| First Electrolyzer 404 | | | |
| CH₄ conv. in SOEC in | 97.7% | 97% | N/A |
| Second Electrolyzer | | | |
| 414 | | | |
| SOEC power | 336 (7 by | 356 | 484 |
| consumption (MW) in | Second | | |
| First Electrolyzer 404, | Electrolyzer 414) | | |
| and Second | (329 by First | | |
| Electrolyzer 414 | Electrolyzer 404) | | |
| Methanol Production | 12 kBPD | 12 kBPD | 12 kBPD |
| from Reactor Unit 422 | | | |

In this process, there are two SOECs (First Electrolyzer 404 and Second Electrolyzer 414), where Second Electrolyzer 414 is the dedicated steam electrolysis SOEC for recycling C₁ and C₂ to the anode, where First Electrolyzer 404 is the main SOEC that does steam and CO₂ electrolysis for syngas production in the cathode. CH₄ was used as a model compound of C₁-C₂ in this simulation example. The performance was summarized in Table 3, Recycle C1 in Anode (corresponding to FIG. 3A). In contrast, the performance is compared against recycling C₁ in the cathode (i.e., instead of sending CH₄ to anode 414-2 of Second Electrolyzer 414, CH₄ was sent to cathode 414-1 of Second Electrolyzer 414 along with steam). The process model of anode CH₄ recycle is established upon the assumption of an equilibrium reactor where the reactions include SMR, POX of CH₄, oxidation of CO and H₂, and water gas shift reaction. A third comparison point is also included where no CH₄ is recycled to the SOEC system at all. In this case, the system operates solely on First Electrolyzer (eliminating Second Electrolyzer) where fresh H₂O and CO₂ feeds was charged at cathode for syngas generation, with no CH₄ recycle. All three configurations are sized to achieve the same final methanol production rate of 12 kBPD, ensuring a consistent production basis for comparison.

At the same CH₄ and CO₂ feed profile (ensuring the same total carbon feed), the simulation showed that at a similar CH₄ conversion rate of 97% to 98%, the total energy consumption of the two SOEC in the anode recycle system was 20 MW less compared to cathode recycle system. Furthermore, when compared to the no-recycle case, the anode recycle mode results in a total power reduction of 148 MW (484 MW vs. 336 MW) at similar H₂O and CO₂ conversion levels. This substantial energy savings highlights that the use of methane in this manner has the potential to enhance the energy efficiency of the system. Firstly, exothermic CH₄ partial oxidation reaction can assist in maintaining the high operating temperature of the SOEC, thereby reducing the need for external heating. Secondly, CH₄ depolarizes the anode by reacting with oxygen ions in the anode to generate electrons, effectively lowering the cell voltage and reducing the electrical power consumption of the SOEC compared to no methane recycle in the anode. In addition, methane reacts with steam to produce syngas (hydrogen and carbon monoxide) via SMR. The heat required for this endothermic reaction can be supplied by the heat generated from the partial oxidation of methane in the SOEC, further helps with the overall energy consumption of the system. In addition, introducing CH₄ also leads to lower H₂O and CO₂ requirements to produce the same methanol output of 12 kBPD, as the recycled CH₄ contributes additional hydrogen and carbon units to the syngas pool. In contrast, the no-recycle configuration demands the highest fresh feed rates to achieve the same methanol production target.

According to an aspect of the present disclosure, a continuous process comprises:
passing one or more of a recycled tail gas stream from a reactor unit and a methane-rich feed stream to an anode of an electrolyzer comprising a cathode, the anode and an electrolyte inserted between the cathode and the anode,
passing one of a steam feed stream or a steam and carbon dioxide feed stream to the cathode of the electrolyzer to produce a cathode effluent comprising one of hydrogen or syngas,
sending a reactor unit product-forming feed stream comprising the cathode effluent to the reactor unit, wherein the reactor unit product-forming feed stream includes at least one of carbon monoxide and carbon dioxide, and
generating, in the reactor unit, a chemical product or a fuel-based, in part, on the reactor unit product-forming feed stream.

According to an aspect of the present disclosure, a system comprises:
an electrolyzer comprising a cathode, an anode and an electrolyte inserted between the cathode and the anode, wherein the electrolyzer is configured to receive one or more of a recycled tail gas stream from a reactor unit and a methane-rich feed stream to the anode and one of a steam feed stream or a steam and carbon dioxide feed stream to the cathode to produce a cathode effluent comprising one of hydrogen or syngas, and
a reactor unit configured to receive a reactor unit product-forming feed stream comprising the cathode effluent and at least one of carbon monoxide and carbon dioxide, and produce a chemical product or a fuel-based, in part, on the reactor unit product-forming feed stream.

According to another aspect of the present disclosure, a continuous process comprises:
generating, in a first electrolyzer, a first cathode effluent comprising hydrogen,
generating, in a second electrolyzer, a second cathode effluent comprising hydrogen, and an anode effluent comprising syngas derived from one or more of a recycled tail gas stream from a reactor unit and a methane-rich feed stream,
sending the first cathode effluent, the second cathode effluent, the anode effluent from the second electrolyzer and a carbon dioxide feed stream to the reactor unit, and
processing, in the reactor unit, the first cathode effluent, the second cathode effluent, the anode effluent from the second electrolyzer and the carbon dioxide feed stream to produce a chemical product or a fuel.

According to another aspect of the present disclosure, a system comprises:
a first electrolyzer comprising a first cathode, a first anode and a first electrolyte inserted between the first cathode and the first anode, wherein the first electrolyzer is configured to receive a first steam feed stream to the cathode to produce a first cathode effluent comprising hydrogen,
a second electrolyzer comprising a second cathode, a second anode and a second electrolyte inserted between the second cathode and the second anode, wherein the second electrolyzer is configured to receive a second steam feed stream to the second cathode to produce a second cathode effluent comprising hydrogen and to receive a third steam feed stream and one or more of a recycled tail gas stream from a reactor unit and a methane-rich feed stream to the anode to produce an anode effluent comprising hydrogen and carbon monoxide, and
a reactor unit configured to receive the first cathode effluent, the second cathode effluent, the anode effluent from the second electrolyzer and a carbon dioxide feed stream, and generate a chemical product or a fuel.

According to another aspect of the present disclosure, a continuous process comprises:
generating, in a first electrolyzer, a first cathode effluent comprising syngas,
generating, in a second electrolyzer, a second cathode effluent comprising syngas, and an anode effluent comprising syngas derived from one or more of a recycled tail gas stream from a reactor unit and a methane-rich feed stream, and
processing, in the reactor unit, the first cathode effluent, the second cathode effluent, and the anode effluent from the second electrolyzer to produce a chemical product or a fuel.

According to another aspect of the present disclosure, a system comprises:
a first electrolyzer comprising a first cathode, a first anode and a first electrolyte inserted between the first cathode and the first anode, wherein the first electrolyzer is configured to receive a first steam feed stream and a first carbon dioxide feed stream to the cathode to produce a first cathode effluent comprising syngas,
a second electrolyzer comprising a second cathode, a second anode and a second electrolyte inserted between the second cathode and the second anode, wherein the second electrolyzer is configured to receive a second steam feed stream and a second carbon dioxide feed stream to the second cathode to produce a second cathode effluent comprising syngas and to receive a third steam feed stream and one or more of a recycled tail gas stream from a reactor unit and a methane-rich feed stream to the anode to produce an anode effluent comprising hydrogen and carbon monoxide, and
a reactor unit configured to receive the first cathode effluent, the second cathode effluent and the anode effluent from the second electrolyzer and generate a chemical product or a fuel.

According to another aspect of the present disclosure, a continuous process comprises:
generating, in a first electrolyzer, a first cathode effluent comprising syngas,
generating, in a second electrolyzer, a second cathode effluent comprising hydrogen, and an anode effluent comprising syngas derived from one or more of a recycled tail gas stream from a reactor unit and a methane-rich feed stream, and
processing, in the reactor unit, the first cathode effluent, the second cathode effluent, and the anode effluent from the second electrolyzer to produce a chemical product or a fuel.

According to another aspect of the present disclosure, a system comprises:
a first electrolyzer comprising a first cathode, a first anode and a first electrolyte inserted between the first cathode and the first anode, wherein the first electrolyzer is configured to receive a first steam feed stream and a carbon dioxide feed stream to the cathode to produce a first cathode effluent comprising syngas,
a second electrolyzer comprising a second cathode, a second anode and a second electrolyte inserted between the second cathode and the second anode, wherein the second electrolyzer is configured to receive a second steam feed stream to the second cathode to produce a second cathode effluent comprising hydrogen and to receive a third steam feed stream and one or more of a recycled tail gas stream from a reactor unit and a methane-rich feed stream to the anode to produce an anode effluent comprising hydrogen and carbon monoxide, and
a reactor unit configured to receive the first cathode effluent, the second cathode effluent and the anode effluent from the second electrolyzer and generate a chemical product or a fuel.

According to another aspect of the present disclosure, a continuous process comprises:
generating, in an electrolyzer, a cathode effluent comprising hydrogen utilizing a methane containing stream comprising one or more of a recycled tail gas stream from a reactor unit and a methane-rich feed stream as fuel in an anode of the electrolyzer, and
processing, in the reactor unit, the cathode effluent and carbon dioxide feed stream to produce a chemical product or a fuel.

According to another aspect of the present disclosure, a system comprises:
an electrolyzer comprising a cathode, an anode and an electrolyte inserted between the cathode and the anode, wherein the electrolyzer is configured to receive a steam feed stream to the cathode and to receive one or more of a recycled tail gas stream from a reactor unit and a methane-rich feed stream to the anode as an energy source for electrolysis to produce a cathode effluent comprising hydrogen,
wherein the reactor unit is further configured to receive the cathode effluent and a carbon dioxide feed stream to produce a chemical product or a fuel.

According to another aspect of the present disclosure, a continuous process comprises:
generating, in an electrolyzer, a cathode effluent comprising syngas utilizing a methane containing stream comprising one or more of a recycled tail gas stream from a reactor unit and a methane-rich feed stream as fuel in an anode of the electrolyzer, and
processing, in the reactor unit, the cathode effluent to produce a chemical product or a fuel.

According to another aspect of the present disclosure, a system comprises:
an electrolyzer comprising a cathode, an anode and an electrolyte inserted between the cathode and the anode, wherein the electrolyzer is configured to receive a steam feed stream and a carbon dioxide feed stream to the cathode and to receive one or more of a recycled tail gas stream from a reactor unit and a methane-rich feed stream to the anode to transfer heat to the cathode to produce a cathode effluent comprising syngas,
wherein the reactor unit is configured to receive the cathode effluent to produce a chemical product or a fuel.

According to an aspect of the present disclosure, a continuous method comprises:
passing a first steam feed stream to a cathode of an electrolyzer comprising the cathode, an anode and an electrolyte inserted between the cathode and the anode, thereby producing a cathode effluent comprising hydrogen,
passing a second steam feed stream and one or more of a recycled tail gas stream from a reactor unit and a methane-rich feed stream to the anode of the electrolyzer, wherein the one or more of the recycled tail gas stream and the methane-rich feed stream are utilized as fuel for producing the cathode effluent comprising hydrogen, and
passing the cathode effluent comprising hydrogen and a carbon dioxide feed stream to the reactor unit, thereby producing a chemical product or a fuel-based product.

In one or more additional illustrative embodiments, as may be combined with the preceding paragraphs, the electrolyzer is operated in a steam electrolysis mode.

In one or more additional illustrative embodiments, as may be combined with the preceding paragraphs, passing the second steam feed stream and one or more of a recycled tail gas stream from a reactor unit and a methane-rich feed stream to the anode of the electrolyzer comprises passing the second steam feed stream, the recycled tail gas stream and the methane-rich feed stream to the anode of the electrolyzer.

In one or more additional illustrative embodiments, as may be combined with the preceding paragraphs, passing the second steam feed stream and one or more of a recycled tail gas stream from a reactor unit and a methane-rich feed stream to the anode of the electrolyzer comprises passing the second steam feed stream and the recycled tail gas stream to the anode of the electrolyzer.

In one or more additional illustrative embodiments, as may be combined with the preceding paragraphs, passing the second steam feed stream and one or more of a recycled tail gas stream from a reactor unit and a methane-rich feed stream to the anode of the electrolyzer comprises passing the second steam feed stream and the methane-rich feed stream to the anode of the electrolyzer.

In one or more additional illustrative embodiments, as may be combined with the preceding paragraphs, the chemical product is one or more of methanol and dimethyl ether.

In one or more additional illustrative embodiments, as may be combined with the preceding paragraphs, the fuel-based product is a Fischer-Tropsch product.

In one or more additional illustrative embodiments, as may be combined with the preceding paragraphs, passing the cathode effluent comprising hydrogen and the carbon dioxide feed stream to the reactor unit further produces a tail gas stream, and the continuous method further comprises:
recycling the tail gas stream to the anode of the electrolyzer.

According to another aspect of the present disclosure, a continuous method comprises:
passing a steam and carbon dioxide feed stream to a cathode of an electrolyzer comprising the cathode, an anode and an electrolyte inserted between the cathode and the anode, thereby producing a cathode effluent comprising syngas,
passing a steam feed stream and one or more of a recycled tail gas stream from a reactor unit and a methane-rich feed stream to the anode of the electrolyzer, wherein the one or more of the recycled tail gas stream and the methane-rich feed stream are utilized as fuel for producing the cathode effluent comprising syngas, and
passing the cathode effluent comprising syngas to the reactor unit, thereby producing a chemical product or a fuel-based product.

In one or more additional illustrative embodiments, as may be combined with the preceding paragraphs, the electrolyzer is operated in a co-electrolysis mode.

In one or more additional illustrative embodiments, as may be combined with the preceding paragraphs, passing the steam feed stream and one or more of a recycled tail gas stream from a reactor unit and a methane-rich feed stream to the anode of the electrolyzer comprises passing the steam feed stream, the recycled tail gas stream and the methane-rich feed stream to the anode of the electrolyzer.

In one or more additional illustrative embodiments, as may be combined with the preceding paragraphs, passing the steam feed stream and one or more of a recycled tail gas stream from a reactor unit and a methane-rich feed stream to the anode of the electrolyzer comprises passing the steam feed stream and the recycled tail gas stream to the anode of the electrolyzer.

In one or more additional illustrative embodiments, as may be combined with the preceding paragraphs, passing the steam feed stream and one or more of a recycled tail gas stream from a reactor unit and a methane-rich feed stream to the anode of the electrolyzer comprises passing the steam feed stream and the methane-rich feed stream to the anode of the electrolyzer.

In one or more additional illustrative embodiments, as may be combined with the preceding paragraphs, the chemical product is one or more of methanol and dimethyl ether.

In one or more additional illustrative embodiments, as may be combined with the preceding paragraphs, the fuel-based product is a Fischer-Tropsch product.

In one or more additional illustrative embodiments, as may be combined with the preceding paragraphs, passing the cathode effluent comprising syngas to the reactor unit further produces a tail gas stream, and the continuous method further comprises:
recycling the tail gas stream to the anode of the electrolyzer.

According to yet another aspect of the present disclosure, a system comprises:
an electrolyzer comprising a cathode, an anode and an electrolyte inserted between the cathode and the anode, wherein the electrolyzer is configured to generate, in the cathode, one of hydrogen from a steam feed stream or syngas from a steam and carbon dioxide feed stream; wherein the electrolyzer is further configured to receive one or more of a recycled tail gas stream and a methane-rich feed stream in the anode utilized as fuel for producing the hydrogen or the syngas in the cathode, and
a reactor unit configured to generate a chemical product or a fuel-based product from the hydrogen or the syngas and a carbon dioxide feed stream.

In one or more additional illustrative embodiments, as may be combined with the preceding paragraphs, the electrolyzer is operated in a steam electrolysis mode when receiving the steam feed stream or in a co-electrolysis mode when receiving the steam and carbon dioxide feed stream.

In one or more additional illustrative embodiments, as may be combined with the preceding paragraphs, the chemical product is one or more of methanol and dimethyl ether.

In one or more additional illustrative embodiments, as may be combined with the preceding paragraphs, the fuel-based product is a Fischer-Tropsch product.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

What is claimed is:

1. A continuous method, comprising:
passing a first steam feed stream to a cathode of an electrolyzer comprising the cathode, an anode and an electrolyte inserted between the cathode and the anode, thereby producing a cathode effluent comprising hydrogen;
passing a second steam feed stream and one or more of a recycled tail gas stream from a reactor unit and a methane-rich feed stream to the anode of the electrolyzer, wherein the one or more of the recycled tail gas stream and the methane-rich feed stream are utilized as fuel for producing the cathode effluent comprising hydrogen; and
passing the cathode effluent comprising hydrogen and a carbon dioxide feed stream to the reactor unit, thereby producing a chemical product or a fuel-based product.

2. The continuous method according to claim 1, wherein the electrolyzer is operated in a steam electrolysis mode.

3. The continuous method according to claim 1, wherein passing the second steam feed stream and one or more of a recycled tail gas stream from a reactor unit and a methane-rich feed stream to the anode of the electrolyzer comprises passing the second steam feed stream, the recycled tail gas stream and the methane-rich feed stream to the anode of the electrolyzer.

4. The continuous method according to claim 1, wherein passing the second steam feed stream and one or more of a recycled tail gas stream from a reactor unit and a methane-rich feed stream to the anode of the electrolyzer comprises passing the second steam feed stream and the recycled tail gas stream to the anode of the electrolyzer.

5. The continuous method according to claim 1, wherein passing the second steam feed stream and one or more of a recycled tail gas stream from a reactor unit and a methane-rich feed stream to the anode of the electrolyzer comprises passing the second steam feed stream and the methane-rich feed stream to the anode of the electrolyzer.

6. The continuous method according to claim 1, wherein the chemical product is one or more of methanol and dimethyl ether.

7. The continuous method according to claim 1, wherein the fuel-based product is a Fischer-Tropsch product.

8. The continuous method according to claim 1, wherein passing the cathode effluent comprising hydrogen and the carbon dioxide feed stream to the reactor unit further produces a tail gas stream, and the continuous method further comprises:
recycling the tail gas stream to the anode of the electrolyzer.

9. A continuous method, comprising:
passing a steam and carbon dioxide feed stream to a cathode of an electrolyzer comprising the cathode, an anode and an electrolyte inserted between the cathode and the anode, thereby producing a cathode effluent comprising syngas;
passing a steam feed stream and one or more of a recycled tail gas stream from a reactor unit and a methane-rich feed stream to the anode of the electrolyzer, wherein the one or more of the recycled tail gas stream and the methane-rich feed stream are utilized as fuel for producing the cathode effluent comprising syngas; and
passing the cathode effluent comprising syngas to the reactor unit, thereby producing a chemical product or a fuel-based product.

10. The continuous method according to claim 9, wherein the electrolyzer is operated in a co-electrolysis mode.

11. The continuous method according to claim 9, wherein passing the steam feed stream and one or more of a recycled tail gas stream from a reactor unit and a methane-rich feed stream to the anode of the electrolyzer comprises passing the steam feed stream, the recycled tail gas stream and the methane-rich feed stream to the anode of the electrolyzer.

12. The continuous method according to claim 9, wherein passing the steam feed stream and one or more of a recycled tail gas stream from a reactor unit and a methane-rich feed stream to the anode of the electrolyzer comprises passing the steam feed stream and the recycled tail gas stream to the anode of the electrolyzer.

13. The continuous method according to claim 9, wherein passing the steam feed stream and one or more of a recycled tail gas stream from a reactor unit and a methane-rich feed stream to the anode of the electrolyzer comprises passing the steam feed stream and the methane-rich feed stream to the anode of the electrolyzer.

14. The continuous method according to claim 9, wherein the chemical product is one or more of methanol and dimethyl ether.

15. The continuous method according to claim 9, wherein the fuel-based product is a Fischer-Tropsch product.

16. The continuous method according to claim 9, wherein passing the cathode effluent comprising syngas to the reactor unit further produces a tail gas stream, and the continuous method further comprises:
recycling the tail gas stream to the anode of the electrolyzer.

17. A system, comprising:
an electrolyzer comprising a cathode, an anode and an electrolyte inserted between the cathode and the anode, wherein the electrolyzer is configured to generate, in the cathode, one of hydrogen from a steam feed stream or syngas from a steam and carbon dioxide feed stream; wherein the electrolyzer is further configured to receive one or more of a recycled tail gas stream and a methane-rich feed stream in the anode utilized as fuel for producing the hydrogen or the syngas in the cathode; and
a reactor unit configured to generate a chemical product or a fuel-based product from the hydrogen or the syngas and a carbon dioxide feed stream.

18. The system according to claim 17, wherein the electrolyzer is operated in a steam electrolysis mode when receiving the steam feed stream or in a co-electrolysis mode when receiving the steam and carbon dioxide feed stream.

19. The system according to claim 17, wherein the chemical product is one or more of methanol and dimethyl ether.

20. The system according to claim 17, wherein the fuel-based product is a Fischer-Tropsch product.
